(19) United States
(12) Patent Application Publication
(10) Pub. No.: US 2016/0177164 A1
(43) Pub. Date: Jun. 23, 2016

(54) Oleaginous Microbial Lubricants

(71) Applicant: Solazyme, Inc., South San Francisco, CA (US)

(72) Inventors: Harrison F. DILLON, South San Francisco, CA (US); Frederyk NGANTUNG, South San Francisco, CA (US); Ana Teresita ECHANIZ, South San Francisco, CA (US)

(73) Assignee: Solazyme, Inc., South San Francisco, CA (US)

(21) Appl. No.: 15/043,420

(22) Filed: Feb. 12, 2016

Related U.S. Application Data
(63) Continuation of application No. 14/200,017, filed on Mar. 7, 2014, now abandoned.
(60) Provisional application No. 61/775,416, filed on Mar. 8, 2013.
(60) Provisional application No. 61/817,793, filed on Apr. 30, 2013.
(60) Provisional application No. 61/829,889, filed on May 31, 2013.
(60) Provisional application No. 61/841,212, filed on Jun. 28, 2013.
(60) Provisional application No. 61/879,676, filed on Sep. 19, 2013.
(60) Provisional application No. 61/914,336, filed on Dec. 10, 2013.
(60) Provisional application No. 61/926,036, filed on Jan. 10, 2014.

(51) Int. Cl. C09K 8/08; C12N 1/12; E21B 21/06; E21B 7/00; E21B 43/16; E21B 43/26; C10M 109/00; C12N 1/16

(52) U.S. Cl. CPC C09K 8/08; C10M 109/00; C12N 1/12; C12N 1/16; E21B 7/00; E21B 43/16; E21B 43/26; E21B 21/062; C09K 2208/34

(57) ABSTRACT

Provided are drilling fluids having delay-released lubrication, the drilling fluids comprising a drilling mud and an oleaginous microbial cell, methods of using and making such drilling fluids, and drilling rigs comprising such drilling fluids. Also provided are lubricants comprising an oleaginous microbial cell. Uses for the lubricants include metal working and extreme pressure applications.

### CROSS-REFERENCE TO RELATED APPLICATIONS

This application is a continuation of U.S. application Ser. No. 14/200,017, filed on Mar. 7, 2014, which claims the benefit under 35 U.S.C. 119(e) of US Provisional Patent Application Nos. 61/775,416, filed Mar. 8, 2013; 61/817,793, filed Apr. 30, 2013; 61/829,889, filed May 31, 2013; 61/841,212, filed Jun. 28, 2013; 61/879,676, filed Sep. 19, 2013; 61/914,336, filed Dec. 10, 2013; and 61/926,036, filed Jan. 10, 2014. Each of these applications is incorporated herein by reference in its entirety for all purposes.

### SEQUENCE LISTING

The instant application contains a Sequence Listing which has been submitted electronically in ASCII format and is hereby incorporated by reference in its entirety. Said ASCII copy, created on Apr. 2, 2014, is named SOLAP018US_SL.txt and is 9,439 bytes in size.

### BACKGROUND

In the use of a cutting tool on a workpiece, friction between the tool and the workpiece can cause wear on the tool, hinder the cutting process, lead to slow manufacturing cycles, and negatively affect the quality and finish of the workpiece. Lubricants are typically used to overcome these undesirable effects. In choosing the appropriate lubricants, consideration needs to be given to the compatibility of the lubricant with both the tool and the workpiece and whether the lubricant can operate efficiently under the conditions of the cutting process. One must also consider the environmental impact of the lubricant in its use and disposal, and on the health of workers using the lubricant.

When drilling subterranean formations, drilling fluids serve, in part, to cool and lubricate the drill bit. Drill bits often encounter increasing downhole friction that arise from changes in downhole pressures, changes in the geological makeup of the formation, and changes in the direction of the drilling, especially when drilling a horizontal well. The increases in friction can lead to a reduced rate of penetration and can limit the ability of the drill bit to reach its target destination with accuracy and efficiency. For example, increasing the rotational torque of the drill bit to address increasing frictional changes can lead to corkscrewing of the drill bit from its intended drilling path and can also cause pipe buckling (both helical and sinusoidal). The increase in friction can also accelerate wear on the drill bit, thus resulting in down time and expensive equipment repair and replacement. Accordingly, the performance demands required of the drilling fluid to provide lubricity to the bit increases over the time course of drilling.

However, current methodologies for reducing downhole friction in lateral wells generally involve reactive addition of lubricant products that are broadly acting, that may adversely affect the rheology of the fluid system, or that may dissipate or degrade over time. Lubricity additives to water based muds range from liquid lubricants (e.g., biodiesel, fatty acid ester, polyalpha-olefins) to mechanical lubricants (e.g., glass beads, copolymer beads, graphite). Adding concentrated “pills” of lubricants have a tendency to lose efficacy over time (e.g., due to dilution, sticking to cuttings, loss to the formation). Mechanical lubricants are effective at reducing friction, but may also create issues in data transmission when using mud pulse telemetry systems for measurement while drilling (MWD) tools if they plug the MWD valve. Additionally, recovery and reuse of beads can also be an issue, particularly if they are broken in use.

### SUMMARY

In one aspect, provided is a drilling fluid for providing delay-released lubrication to a drill bit in a drilling operation, the fluid comprising:

a) a drilling mud and

b) an oleaginous microbial cell;

said fluid capable of providing increasing lubricity during drilling and one or more of

i) at least a 5% reduction, e.g., at least a 10%, 15%, 20%, 25% reduction, in torque to the drill string;

ii) at least a 5% increase in rate of penetration; or

iii) at least a 5% reduction in drag.

In one aspect, provided is a drilling fluid for providing delay-released lubrication to a drill bit in a drilling operation, the fluid comprising:

a) a drilling mud and

b) an oleaginous microbial cell;

said fluid capable of providing increasing lubricity during drilling and one or more of

i) at least a 5% reduction, e.g., at least a 10%, 15%, 20%, 25% reduction, in torque to the drill bit;

ii) at least a 5% increase in rate of penetration; or

iii) at least a 5% reduction in drag.

In one aspect, provided is a drilling fluid for providing delay-released lubrication to a drill bit in a drilling operation comprising a drilling mud and an oleaginous microbial cell. In varying embodiments, the fluid is capable of providing or provides increasing lubricity during drilling and at least a 5% reduction, e.g., at least a 10%, 15%, 20%, 25% reduction, in torque to the drill bit.

In some embodiments, the fluid is capable of providing or provides increasing lubricity over at least a 5, 15, 30, 45, or 60 minute time period.

In some embodiments, the fluid is capable of providing or provides at least a 25%, 30%, 35%, 40%, 45%, 50%, 55%, 60%, 65%, 70%, or 75% reduction in torque.

In some embodiments, the fluid is capable of providing or provides at least a 60%, 65%, 70%, or 75% reduction in torque.

In one aspect, provided is a method for preparing a drilling fluid for providing delay-released lubrication to a drill bit in a drilling operation, the method comprising mixing a drilling mud with an oleaginous microbial cell to form a drilling fluid capable of increasing lubricity during drilling and one or more of

i) at least a 5% reduction, e.g., at least a 10%, 15%, 20%, 25% reduction, in torque to the drill bit;

ii) at least a 5% increase in rate of penetration; or

iii) at least a 5% reduction in drag.

In one aspect, provided is a method for providing delay-released lubrication to a drill bit in a drilling operation, the method comprising mixing a drilling mud with an oleaginous microbial cell to form a drilling fluid capable of increasing lubricity during drilling and reducing torque at the drill bit by at least 20%.

In one aspect, provided is a method for drilling a wellbore in a drilling operation, the method comprising circulating a drilling fluid provided herein through the wellbore.

In some embodiments, the microbial cell is in an amount that is 40%, 35%, 30%, 25%, 20%, 15%, 10%, 5%, 3%, 2%, or 1% or less by volume of the drilling fluid.

In some embodiments, the microbial cell is in an amount that is 10% or less by volume of the drilling fluid.

In some embodiments, the microbial cell is in an amount that is 6% or less by volume of the drilling fluid.

In some embodiments, the microbial cell comprises a microalgal cell containing at least 45%, 50%, 55%, 60%, 65%, 70%, 75%, 80%, or 85% oil.

In some embodiments, the microbial cell comprises a whole cell.

In some embodiments, the microbial cell comprises a lysed cell. In some embodiments the oil has been extracted from the lysed cell to give a de-fatted cell. In some embodiments the lysed, de-fatted cells contain less than 15%, 14%, 13%, 12%, 11%, 10%, 9%, 8%, 7%, 6%, 5%, 4%, 3%, 2%, or 1% oil. In some embodiments the lysed, de-fatted cells are mixed with whole cells. In some embodiments provided is a drilling fluid comprising a mixture lysed, de-fatted cells and whole cells. In some embodiments the amount by weight of lysed, de-fatted cells in the mixture is less than the amount of whole cells. In some embodiments the weight ratio of lysed, de-fatted cells to whole cells in the mixture is no more than 1:30, 1:25, 1:20 1:10, 1:9, 1:8:1, 1:7, 1:6, 1:5, 1:4, 1:3, 1:2, or 1:1. In other embodiments the amount by weight of lysed, de-fatted cells in the mixture is greater than the amount of whole cells. In other embodiments the weight ratio of lysed, de-fatted cells to whole cells in the mixture is at least 20:1, 10:1, 9:1, 8:1, 7:1, 6:1, 5:1, 4:1, 3:1, or 2:1.

In some embodiments, the microbial cell comprises an oleaginous bacteria, yeast, or microalgae.

In some embodiments, the microbial cell is obtained from a heterotrophic oleaginous microalgae.

In some embodiments, the microbial cell is obtained from microalgae cultivated with sugar from corn, sorghum, sugar cane, sugar beet, or molasses as a carbon source.

In some embodiments, the microbial cell is obtained from microalgae cultivated on sucrose.

In some embodiments, the microbial cell is obtained from Parachlorella, Prototheca, or Chlorella.

In some embodiments, the microbial cell is obtained from Prototheca moriformis.

In some embodiments, the microbial cell is an oleaginous microalgae having a fatty acid profile of at least 60% C18:1; or at least 50% combined total amount of C10, C12, and C14; or at least 70% combined total amount of C16:0 and C18:1.

In some embodiments, the drilling mud is a water-based mud, a synthetic-based mud, or an oil-based mud.

In some embodiments, the drilling operation is a land-based or an off-shore drilling operation.

In some embodiments, the drilling operation is selected from the group consisting of completion operations, sand control operations, workover operations, and hydraulic fracturing operations.

In some embodiments, the wellbore is a vertical, horizontal, or deviated wellbore. In some embodiments, the wellbore is a vertical or horizontal wellbore.

In one aspect, provided is a drilling rig containing a drilling fluid provided herein.

In some embodiments, the fluid is in a drill pipe or mud tank.

In some embodiments, a lubricant comprises an oleaginous microbial cell, and the cell containing at least 45% oil by dry cell weight. In some embodiments, the cell contains or comprises at least 50%, 55%, 60%, 65%, 70%, 75%, or 80% oil by dry cell weight.

In some embodiments, the lubricant is capable of providing or provides at least a 25%, 30%, 35%, 40%, 45%, 50%, 55%, 60%, 65%, 70%, or 75% reduction in torque. In some embodiments, the lubricant is capable of providing at least a 60%, 65%, 70%, or 75% reduction in torque.

In some embodiments, the lubricant is capable of providing or provides at least a 5%, 10%, 15%, 20%, 25%, 30%, 35%, 40%, or 45% increase in rate of penetration. In some embodiments the lubricant is capable of providing at least a 20% increase in rate of penetration.

In some embodiments, the lubricant is capable of providing or at least provides at least a 5%, 10%, 15%, 20%, 25%, 30%, 35%, 40%, or 45% reduction in drag. In some embodiments the lubricant is capable of providing at least a 32% reduction in drag.

In some embodiments, the microbial cell comprises a whole cell. In some embodiments, the microbial cell comprises a lysed cell. In some embodiments, the microbial cell comprises an oleaginous bacteria, yeast, or microalgae. In some embodiments, the microbial cell is obtained from a heterotrophic oleaginous microalgae. In some embodiments, the microbial cell is obtained from microalgae cultivated with sugar from corn, sorghum, sugar cane, sugar beet, or molasses as a carbon source. In some embodiments, the microbial cell is obtained from microalgae cultivated on sucrose.

In some embodiments, the microbial cell is obtained from Parachlorella, Prototheca, or Chlorella. In some embodiments, the microbial cell is obtained from Prototheca moriformis.

In some embodiments, the cells are in powdered form. The powdered cells can be in a dry powder form.

In some embodiments provided is a biodegradable lubricant or drilling fluid.

In some embodiments, the microbial cell contains or comprises an oleaginous microalgae having a fatty acid profile of at least 60% C18:1; or at least 50% combined total amount of C10, C12, and C14; or at least 70% combined total amount of C16:0 and C18:1.

In some embodiments, the microbial oil provided herein is a microalgal oil comprising C29 and C28 sterols, wherein the amount of C28 sterols is greater than C29 sterols.

In some embodiments, the microbial oil provided herein is a microalgal oil comprising one or more of: at least 10% ergosterol; ergosterol and β-sitosterol, wherein the ratio of ergosterol to β-sitosterol is greater than 25:1; ergosterol and brassicasterol; ergosterol, brassicasterol, and poriferasterol, and wherein the oil is optionally free from one or more of β-sitosterol, campesterol, and stigmasterol.

In some embodiments, the lubricant is an extreme pressure lubricant.

In some embodiments, provided is a metal working fluid comprising a lubricant provided herein.

In some embodiments, the lubricant is in an amount that is 90%, 80%, 70%, 60%, 50%, 40%, 35%, 30%, 25%, 20%, 15%, 10%, 5%, 3%, 2%, or 1% or less by volume of the fluid.

In some embodiments, the metal working fluid is an insoluble oil, soluble oil, semisynthetic, or synthetic metal working fluid.

In some embodiments, the metal working fluid further comprises one or more of a surfactant, emulsifier, defoamer, alkaline reserve, anti-mist agent, corrosion inhibitor, biocide, extreme pressure additive, coupling agent, thickener, chelating agent, lubricity agent, humectant, odorant, or dye.

In some embodiments, the surfactant comprises an ether, an alkoxylated nonylphenol, or mixtures thereof. In some embodiments, the emulsifier comprises a hexohydrobenzoic acid, naphthenate, sulfonate, soap, amide, nonionic ethoxylate, an amphoteric, or mixtures thereof. In some embodiments, the defoamer comprises a silicone, waxy, calcium nitrite, acetate, or mixtures thereof. In some embodiments, the alkaline reserve comprises an alkanolamine, an alkali hydroxide, or mixtures thereof. In some embodiments, the anti-mist agent comprises a polybutene, polyacrylate, polyethylene oxide, or mixtures thereof. In some embodiments, the corrosion inhibitor comprises an amine carboxylate, amine dicarboxylate, boramide, arylsulfonamido acid, sodium borate, sodium molybdate, sodium metasilicate, succinic acid derivative, tolyltriazole, benzotriazole, benzothiazole, thiadiazole, diethanolamine, triethanolamine, nitrite, chlorophenols, cresol, formaldehyde formalin, iodine, phosphate, organic mercurials, phenols, quarternary ammonium compounds, oxammonium, S-triazine compounds, tris-hydroxymethylnitromethane, or mixtures thereof. In some embodiments, the biocide comprises a triazine, nitromorpholine, polymeric quat, bromonirile, phenol, halogenated carbamate, isothiazolone, or mixtures thereof. In some embodiments, the extreme pressure additive comprises a sulfurized hydrocarbon, sulfurized fatty acid ester, chlorinated paraffin, chlorinated acid, chlorinated ester, phosphate ester, or mixtures thereof. In some embodiments, the coupling agent comprises an alcohol, ether, glycol ether, hexylene glycol, or mixtures thereof. In some embodiments, the thickener comprises a polyether, a polyvinyl alcohol, or mixtures thereof. In some embodiments, the chelating agent comprises sodium EDTA, a phosphonate, gluconate, or mixtures thereof. In some embodiments, the lubricity agent comprises an aromatic oil, esters, naphthenic oil, paraffinic oil, polyether glycol, ester, fatty acid ester, glycol ester, block copolymer, or mixtures thereof. In some embodiments, the humectant comprises a polymeric ether, an ester, or mixtures thereof. In some embodiments, the odorant comprises an aldehyde. In some embodiments, the dye comprises an azo dye, a fluorescein, or mixtures thereof.

In some embodiments the oil encapsulated cells provided herein have an average diameter of about 5 to 10 microns.

In some embodiments the lubricants (e.g. encapsulated cells) provided herein are used as a lubricant in trenchless tunneling operations. Trenchless tunneling methods are desirable for underground installation of utilities such as sewer, water, gas, electricity, and telecommunications in congested areas such as under roadways and city streets, or in soft soils, environmentally sensitive or contaminated areas, or near water crossings, where open cut trench excavation, pipe installation, and subsequent backfill are inconvenient or difficult.

In some embodiments, the lubrication provided herein is in used in a microtunneling operation. In some embodiments, provided is a microtunneling boring machine (MTBM) comprising a lubricant provided herein. In some embodiments the lubricant is for lubricating the interface between the earth and the cutting wheel or between the earth and the pipe section.

In microtunneling, an entry pit is prepared to receive a steerable MTBM that is advanced horizontally towards a receiving pit. The MTBM typically bores tunnels ranging from 1 to 10 feet in diameter, more commonly from 1 to 3 feet. Because of this small diameter, the MTBM is guided by remote control and follows a projected laser beam. The MTBM houses a cutting wheel and optionally a trailing component engaged with a jacking frame. The pipes that are to be installed are positioned behind the cutting wheel or, when present, behind the trailing component. This assembly is pushed by hydraulic jacks mounted on the jacking frame. Slurry feed and discharge lines are connected to the MTBM to allow for removal of cuttings. In some embodiments, the slurry comprises a lubricant provided herein to lubricate the cutting wheel. In some embodiments the slurry further comprises bentonite.

The diameter of the cutting wheel used is typically slightly greater than the diameter of the pipes to create an overcut resulting in an annular space around the pipes. This space reduces frictional forces on the pipes as they are being advanced. Lubricants from the MTBM can be injected into the annular space to further reduce the frictional forces on the pipe/pipestring and to reduce the jacking forces required to advance the pipe/pipestring. Typical lubricants include bentonite, and chemical polymers can also be used. In some embodiments, provided is a lubricant comprising bentonite and an oil encapsulated cell provided herein. Once the pipes have been installed the annular space can be filled with cement grout.

In some embodiments, a slurry containing a lubricant provided herein acts to lubricate the drilling assembly as it contacts and moves against the earth, counterbalances the earth pressures resulting from the excavation, forms a filter cake against the earth to limit fluid losses, facilitates removal of the cuttings from the well/tunnel, and/or facilitates separation of the solid components from the liquid components as the slurry is circulated from the well/tunnel to a separation plant for recycling. In some embodiments the liquid component of the slurry is water. In some embodiments the water has a pH of between 8.0 and 10. In some embodiments the slurry contains bentonite, a bentonite salt, or a combination of the two. In some embodiments the slurry contains sodium montmorillonite. Bentonite containing slurries are particularly beneficial when used in sandy or coarse grained soils with fines content of 50% or less as defined by ASTM D-2487, while non-bentonite based slurries are recommended when fines content are greater than 50%. In some embodiments the slurry is substantially free from bentonite. In some embodiments the slurry contains polymers and/or inert solids.

In some embodiments the drilling fluids provided herein contain oils encapsulated in microbial cells wherein the oils are released when microbial cells when exposed to conditions favorable to cell lysis. Such conditions include temperature, pressure, shear and friction; in the absence of lysing conditions, the cells recirculate through the mud system. The cells are thus able to release its cellular contents and deliver the lubricating oil directly to the area in need of lubrication. The precise delivery of the lubricant at the appropriate time and place maximizes the effectiveness of the lubricant and minimizes waste. In some embodiments the cells encapsulating the oils contain a polysaccharide rich shell. In some embodiments the reduction in friction to the drill string provided by the lubricant allows for improved directional control of the drill bit and for drilling cleaner and straighter holes. The reduction in friction also allows for the drill bit to be drilled further and faster, while reducing stuck pipe instances, tool maintenance, and interval changes.

### BRIEF DESCRIPTION OF THE DRAWINGS

FIG. 1 illustrates coefficients of lubricity of water based mud containing oil from Strains A and B as a function of time and in comparison to mud containing industrial lubricants.

FIG. 2 illustrates coefficients of lubricity of water based mud containing lysed or whole cells from Strains A and B as a function of time and in comparison to mud containing industrial lubricants.

FIG. 3 illustrates coefficients of lubricity of synthetic based mud containing lysed or whole cells from Strains A and B as a function of time and in comparison to mud containing industrial lubricants.

FIG. 4 illustrates coefficients of lubricity of salty water based mud containing lysed or whole cells from Strains A and B as a function of time and in comparison to mud containing industrial lubricants.

FIG. 5 illustrates cell lysis of Strain B cells isolated from broth or that were further drum dried.

FIG. 6 illustrates the drill path for a field trial using water based mud with whole microalgal cells from Strain A in comparison to using water based mud alone.

FIG. 7 illustrates hook weights (lb) of drill bottom housing assemblies provided with water based mud containing whole cells from Strain A as a function of bit height (ft) and in comparison to water based mud alone when tripping out at 1,110-1170 and 1,285-1,330 feet (measured distance) corresponding to 45 and 60 degree portions of the curve.

FIG. 8 illustrates drag measurements at the 60 degree portion of the curve.

FIG. 9 illustrates the rotational torque required to rotate the drill string and bottom hole assembly in the presence and absence of encapsulated oil.

FIG. 10 illustrates rate of penetration observed when drilling laterally in the presence and absence of encapsulated oil.

FIG. 11 illustrates the interaction the encapsulated oils with the bottom hole assembly and formation. 11A) Encapsulated oil is added and circulates throughout the drilling fluid system. 11B) Under the appropriate stimulus (high friction, shear, extreme pressure, etc.) cells containing the oil rupture and oil is released. 11C) Oil is delivered at high effective concentration to lubricate and coat where it is needed. 11D) Unbroken cells are re-circulated throughout the system.

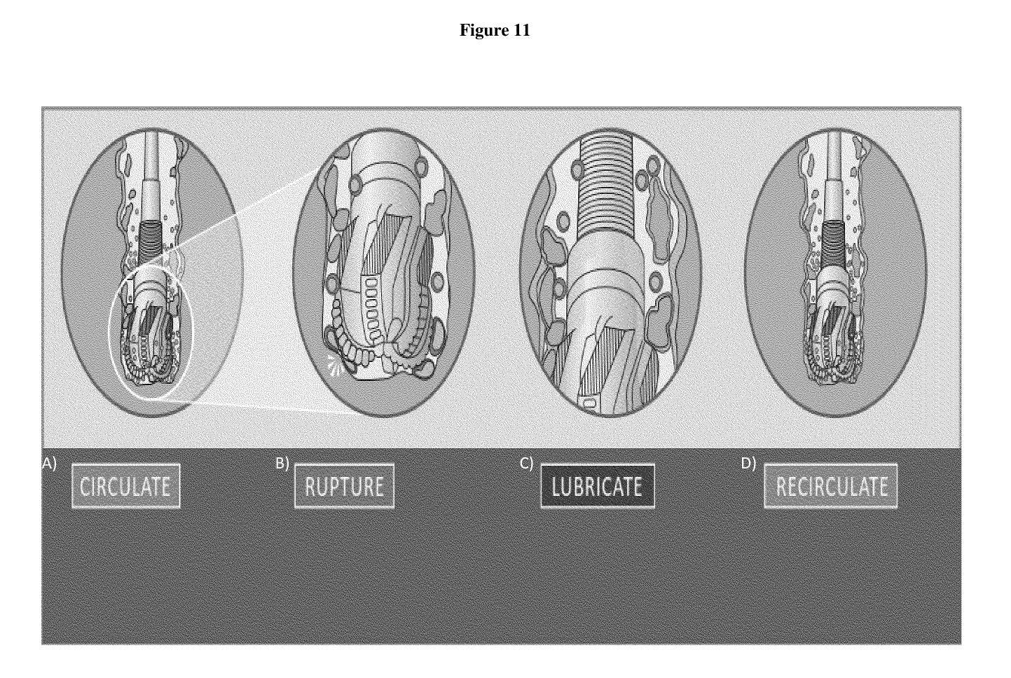

FIG. 12 illustrates the percent cell lysis based on free oil release of microalgal and yeast strains in water at increasing pressures.

FIG. 13 illustrates the reductions in torque observed in water containing microalgal or yeast cells or free oil compared to a petroleum based lubricant (Stabil Lube).

### DETAILED DESCRIPTION

### Definitions

Unless defined otherwise, all technical and scientific terms used herein have the meaning commonly understood by a person skilled in the art to which this invention belongs. As used herein, the following terms have the meanings ascribed to them unless specified otherwise.

“Bottom hole assembly” or “BHA” refers to the portion of the drill string attached to the drill pipe that includes the drill bit as well as drill collar(s) and related assemblies that assist, in part, to provide weight to the bit.

“Biomass” is material produced by growth and/or propagation of cells. Biomass may contain cells and/or intracellular contents as well as extracellular material, includes, but is not limited to, compounds secreted by a cell. Biomass isolated from fermentation broth may include nutrients and feedstock used to grow the cells.

“Bridging material” is material added to a fluid that prevents or decreases loss of the fluid through geologic formations that have pores that are greater than 1 millidarcy.

“Bioreactor” and “fermentor” mean an enclosure or partial enclosure, such as a fermentation tank or vessel, in which cells are cultured, typically in suspension.

“Cellulosic material” includes the product of digestion of cellulose, including glucose and xylose, and optionally additional compounds such as disaccharides, oligosaccharides, lignin, furfurals and other compounds. Nonlimiting examples of sources of cellulosic material include sugar cane bagasses, sugar beet pulp, corn stover, wood chips, sawdust and switchgrass.

“Cultivated”, and variants thereof such as “cultured” and “fermented”, refer to the intentional fostering of growth (increases in cell size, cellular contents, and/or cellular activity) and/or propagation (increases in cell numbers via mitosis) of one or more cells by use of selected and/or controlled conditions. The combination of both growth and propagation is termed proliferation. Examples of selected and/or controlled conditions include the use of a defined medium (with known characteristics such as pH, ionic strength, and carbon source), specified temperature, oxygen tension, carbon dioxide levels, and growth in a bioreactor. Cultivate does not refer to the growth or propagation of microorganisms in nature or otherwise without human intervention; for example, natural growth of an organism that ultimately becomes fossilized to produce geological crude oil is not cultivation.

“Dry weight” and “dry cell weight” mean weight determined in the relative absence of water. For example, reference to oleaginous yeast biomass as comprising a specified percentage of a particular component by dry weight means that the percentage is calculated based on the weight of the biomass after substantially all water has been removed.

“Exogenous gene” is a nucleic acid that codes for the expression of an RNA and/or protein that has been introduced (“transformed”) into a cell. A transformed cell may be referred to as a recombinant cell, into which additional exogenous gene(s) may be introduced. The exogenous gene may be from a different species (and so heterologous), or from the same species (and so homologous), relative to the cell being transformed. Thus, an exogenous gene can include a homologous gene that occupies a different location in the genome of the cell or is under different control, relative to the endogenous copy of the gene. An exogenous gene may be present in more than one copy in the cell. An exogenous gene may be maintained in a cell as an insertion into the genome or as an episomal molecule.

“Fixed carbon source” is a molecule(s) containing carbon, typically an organic molecule, that is present at ambient temperature and pressure in solid or liquid form in a culture media that can be utilized by a microorganism cultured therein.

“Fluid loss control agent” is material added to a fluid that prevents or decreases loss of the liquid component of the fluid through geologic formations that have pores that are less than 1 millidarcy.

“Growth” means an increase in cell size, total cellular contents, and/or cell mass or weight of an individual cell, including increases in cell weight due to conversion of a fixed carbon source into intracellular oil.

“Homogenate” is biomass that has been physically disrupted.

“Limiting concentration of a nutrient” is a concentration of a compound in a culture that limits the propagation of a cultured organism. A “non-limiting concentration of a nutrient” is a concentration that supports maximal propagation during a given culture period. Thus, the number of cells produced during a given culture period is lower in the presence of a limiting concentration of a nutrient than when the nutrient is non-limiting. A nutrient is said to be “in excess” in a culture, when the nutrient is present at a concentration greater than that which supports maximal propagation.

“Lipids” are a class of molecules that are soluble in nonpolar solvents (such as ether and chloroform) and are relatively or completely insoluble in water. Lipid molecules have these properties, because they consist largely of long hydrocarbon chains which are hydrophobic in nature. Examples of lipids include fatty acids (saturated and unsaturated); glycerides or glycerolipids (such as monoglycerides, diglycerides, triglycerides or neutral fats, and phosphoglycerides or glycerophospholipids); nonglycerides (sphingolipids, sterol lipids including cholesterol and steroid hormones, prenol lipids including terpenoids, fatty alcohols, waxes, and polyketides); and complex lipid derivatives (sugar-linked lipids, or glycolipids, and protein-linked lipids). “Fats” or “triglyceride oils” are a subgroup of lipids called “triacylglycerides.” The fatty acids are conventionally named by the notation that recites number of carbon atoms and the number of double bonds separated by a colon. For example oleic acid can be referred to as C18:1 and capric acid can be referred to as C10:0. As used herein, the term “triacylglycerides” and “triglycerides” are interchangeable.

“Lubricity” refers to the ability of a lubricant to reduce frictional forces such as torque and drag forces acting on a drill bit or drill string. The lubricity of a lubricant is measured by its coefficient of friction, which is defined as the ratio of the force required to move an object to the force applied perpendicular to the object. A low coefficient of friction corresponds to high lubricity.

“Lysate” is a solution containing the contents of lysed cells.

“Lysis” is the breakage of the plasma membrane and optionally the cell wall of a biological organism sufficient to release at least some intracellular content, often by mechanical, viral or osmotic mechanisms that compromise its integrity.

“Lysing” is disrupting the cellular membrane and optionally the cell wall of a biological organism or cell sufficient to release at least some intracellular content.

“Microorganism” and “microbe” are microscopic unicellular organisms.

“Mud” or “drilling fluid” is a generic term used to refer to a fluid used in drilling operations. Drilling fluids typically perform a number of functions, including cooling and lubricating the drill bit and drill string, transporting cuttings from the drill bit to the surface, and controlling downhole pressures to prevent blow-outs. Examples of drilling fluids include water based drilling fluids and non-aqueous based systems such as oil based and synthetic based drilling fluids.

“Oil” means any triacylglyceride (or triglyceride oil), produced by organisms, including oleaginous yeast, plants, and/or animals. “Oil,” as distinguished from “fat”, refers, unless otherwise indicated, to lipids that are generally liquid at ordinary room temperatures and pressures. For example, “oil” includes vegetable or seed oils derived from plants, including without limitation, an oil derived from soy, rapeseed, canola, palm, palm kernel, coconut, corn, olive, sunflower, cotton seed, cuphea, peanut, camelina sativa, mustard seed, cashew nut, oats, lupine, kenaf, calendula, hemp, coffee, linseed, hazelnut, euphorbia, pumpkin seed, coriander, camellia, sesame, safflower, rice, tung oil tree, cocoa, copra, opium poppy, castor beans, pecan, jojoba, jatropha, macadamia, Brazil nuts, and avocado, as well as combinations thereof.

“Oleaginous microorganism”, “oleaginous microbe”, and “oleaginous microbial cell” refers to a microorganism or cell producing at least 20% lipid by dry cell weight. The microorganisms include wild-type, genetically engineered, or mutated microorganisms. In particular embodiments, the microorganism yields cells that are at least 40%, at least 45%, at least 50%, at least 55%, at least 60%, at least 65%, or at least 70% or more lipid. “Oleaginous yeast” means yeast that can naturally accumulate more than 20% of their dry cell weight as lipid and are of the Dikarya subkingdom of fungi. Oleaginous yeast includes organisms such as Yarrowia lipolytica, Rhodotorula glutinis, Cryptococcus curvatus and Lipomyces starkeyi.

“Polysaccharides” or “glycans” are carbohydrates made up of monosaccharides joined together by glycosidic linkages. Cellulose is a polysaccharide that makes up certain plant cell walls. Cellulose can be depolymerized by enzymes to yield monosaccharides such as xylose and glucose, as well as larger disaccharides and oligosaccharides.

“Predominantly encapsulated” means that more than 50% of a referenced component, e.g., algal oil, is sequestered in an oleaginous microbe cell or cells.

“ppb” refers to pounds per barrel. 1 ppb is equivalent to 1 gram material per 350 mL base fluid.

“Predominantly intact cells” and “predominantly intact biomass” mean a population of cells that comprise more than 50% intact cells. “Intact”, in this context, means that the physical continuity of the cellular membrane and/or cell wall enclosing the intracellular components of the cell has not been disrupted in any manner that would release the intracellular components of the cell to an extent that exceeds the permeability of the cellular membrane in culture.

“Predominantly lysed” means a population of cells in which more than 50%, of the cells have been disrupted such that the intracellular components of the cell are no longer completely enclosed within the cell membrane.

A “fatty acid profile” is the distribution of fatty acyl groups in the triglycerides of the oil without reference to attachment to a glycerol backbone. Fatty acid profiles are typically determined by conversion to a fatty acid methyl ester (FAME), followed by gas chromatography (GC) analysis with flame ionization detection (FID). The fatty acid profile can be expressed as one or more percent of a fatty acid in the total fatty acid signal determined from the area under the curve for that fatty acid. FAME-GC-FID measurement approximate weight percentages of the fatty acids. A “sn-2 profile” is the distribution of fatty acids found at the sn-2 position of the triacylglycerides in the oil. A “regiospecific profile” is the distribution of triglycerides with reference to the positioning of acyl group attachment to the glycerol backbone without reference to stereospecificity. In other words, a regiospecific profile describes acyl group attachment at sn-⅓ vs. sn-2. Thus, in a regiospecific profile, POS (palmitate-oleate-stearate) and SOP (stearate-oleate-palmitate) are treated identically. A “stereospecific profile” describes the attachment of acyl groups at sn-1, sn-2 and sn-3. Unless otherwise indicated, triglycerides such as SOP and POS are to be considered equivalent. A “TAG profile” is the distribution of fatty acids found in the triglycerides with reference to connection to the glycerol backbone, but without reference to the regiospecific nature of the connections. Thus, in a TAG profile, the percent of SSO in the oil is the sum of SSO and SOS, while in a regiospecific profile, the percent of SSO is calculated without inclusion of SOS species in the oil. In contrast to the weight percentages of the FAME-GC-FID analysis, triglyceride percentages are typically given as mole percentages; that is the percent of a given TAG molecule in a TAG mixture.

“Proliferation” means a combination of both growth and propagation.

“Propagation” means an increase in cell number via mitosis or other cell division.

“Renewable diesel” is a mixture of alkanes (such as C10:0, C12:0, C14:0, C16:0 and C18:0) produced through hydrogenation and deoxygenation of lipids.

“Spent biomass” and variants thereof such as “delipidated meal” and “defatted biomass” is microbial biomass after oil (including lipids) and/or other components have been extracted or isolated from it; e.g., through the use of mechanical (i.e., exerted by an expeller press) or solvent extraction or both. Such delipidated meal has a reduced amount of oil/lipids as compared to before the extraction or isolation of oil/lipids from the microbial biomass but typically contains some residual oil/lipid.

“Sonication” is a process of disrupting biological materials, such as a cell, by use of sound wave energy.

“Viscosity modifying agent” is an agent that modifies the rheological properties of a fluid. The viscosity of a fluid is the measure of the resistance of a fluid to flow. The viscosity modifying agent is used to increase or decrease the viscosity of a fluid used in oil field chemical applications

“V/V” or “v/v”, in reference to proportions by volume, means the ratio of the volume of one substance in a composition to the volume of the composition. For example, reference to a composition that comprises 5% v/v yeast oil means that 5% of the composition's volume is composed of oil (e.g., such a composition having a volume of 100 mm³ would contain 5 mm³ of oil), and the remainder of the volume of the composition (e.g., 95 mm³ in the example) is composed of other ingredients.

“W/V” or “w/v”, in reference to a concentration of a substance means grams of

“W/W” or “w/w”, in reference to proportions by weight, means the ratio of the weight of one substance in a composition to the weight of the composition. For example, reference to a composition that comprises 5% w/w oleaginous yeast biomass means that 5% of the composition's weight is composed of oleaginous yeast biomass (e.g., such a composition having a weight of 100 mg would contain 5 mg of oleaginous yeast biomass) and the remainder of the weight of the composition (e.g., 95 mg in the example) is composed of other ingredients.

### Oleaginous Microbes and Heterotrophic Culture Conditions

The triacylglycerides used in the preparation of the triacylglyeride mixtures can be obtained from any organism producing triacylglycerides with C18:1 or saturated C:4-C24 fatty acids. Production of hydrocarbons by microorganisms is reviewed by Metzger et al., Appl Microbiol Biotechnol (2005) 66: 486-496 and A Look Back at the U.S. Department of Energy's Aquatic Species Program: Biodiesel from Algae, NREL/TP-580-24190, John Sheehan, Terri Dunahay, John Benemann and Paul Roessler (1998), incorporated herein by reference.

The triacylglycerides used in the preparation of the triacylglyeride mixtures can be obtained from any organism producing triacylglycerides with C18:1 or saturated C4-C24 fatty acids. Production of hydrocarbons by microorganisms is reviewed by Metzger et al., Appl Microbiol Biotechnol (2005) 66: 486-496 and A Look Back at the U.S. Department of Energy's Aquatic Species Program: Biodiesel from Algae, NREL/TP-580-24190, John Sheehan, Terri Dunahay, John Benemann and Paul Roessler (1998), incorporated herein by reference.

In particular embodiments, the microorganism yields cells that are at least: about 40%, to 60% or more (including more than 70%) lipid when harvested for oil extraction. For many applications, organisms that grow heterotrophically (on sugar or a carbon source other than carbon dioxide in the absence of light) or can be engineered to do so, are useful in the methods and drilling fluids provided herein. See PCT Publication Nos. 2010/063031; 2010/063032; 2008/151149, each of which is incorporated herein by reference in their entireties.

Naturally occurring and genetically engineered microalgae are suitable microorganisms as sources of C18:1 or saturated C4-C24 triacylglycerides suitable for use in the methods and materials provided herein. Thus, in various embodiments, the microorganism from which the triacylglyceride is obtained is a microalgae. Examples of genera and species of microalgae include, but are not limited to, the following genera and species microalgae in Table 1.

TABLE 1
Microalgae

Achnanthes orientalis, Agmenellum, Amphiprora hyaline, Amphora coffeiformis, Amphora
coffeiformis linea, Amphora coffeiformis punctata , Amphora coffeiformis taylori , Amphora
coffeiformis tenuis, Amphora delicatissima, Amphora delicatissima capitata , Amphora sp.,
Anabaena, Ankistrodesmus, Ankistrodesmus falcatus, Boekelovia hooglandii , Borodinella
sp., Botryococcus braunii, Botryococcus sudeticus, Bracteoccocus aerius, Bracteococcus
sp., Bracteacoccus grandis, Bracteacoccus cinnabarinas, Bracteococcus minor,
Bracteococcus medionucleatus, Carteria, Chaetoceros gracilis, Chaetoceros muelleri,
Chaetoceros muelleri subsalsum, Chaetoceros sp., Chlorella anitrata, Chlorella Antarctica,
Chlorella aureoviridis, Chlorella candida, Chlorella capsulate, Chlorella desiccate,
Chlorella ellipsoidea, Chlorella emersonii, Chlorella fusca, Chlorella fusca var. vacuolata,
Chlorella glucotropha, Chlorella infusionum, Chlorella infusionum var. actophila,
Chlorella infusionum var. auxenophila, Chlorella kessleri, Chlorella lobophora (strain
SAG 37.88), Chlorella luteoviridis, Chlorella luteoviridis var. aureoviridis, Chlorella
luteoviridis var. lutescens, Chlorella miniata, Chlorella cf. minutissima, Chlorella
minutissima, Chlorella mutabilis, Chlorella nocturna, Chlorella ovalis, Chlorella parva,
Chlorella photophila, Chlorella pringsheimii, Chlorella protothecoides (including any of
UTEX strains 1806, 411, 264, 256, 255, 250, 249, 31, 29, 25), Chlorella protothecoides var.
acidicola, Chlorella regularis, Chlorella regularis var. minima, Chlorella regularis var.
umbricata, Chlorella reisiglii, Chlorella saccharophila, Chlorella saccharophila var.
ellipsoidea, Chlorella salina, Chlorella simplex, Chlorella sorokiniana, Chlorella sp.,
Chlorella sphaerica, Chlorella stigmatophora, Chlorella vanniellii, Chlorella vulgaris,
Chlorella vulgaris f. tertia, Chlorella vulgaris var. autotrophica, Chlorella vulgaris var.
viridis, Chlorella vulgaris var. vulgaris, Chlorella vulgaris var. vulgaris f. tertia,
Chlorella vulgaris var. vulgaris f. viridis, Chlorella xanthella, Chlorella zofingiensis,
Chlorella trebouxioides, Chlorella vulgaris, Chlorococcum infusionum, Chlorococcum sp.,
Chlorogonium, Chroomonas sp., Chrysosphaera sp., Cricosphaera sp., Crypthecodinium
cohnii, Cryptomonas sp., Cyclotella cryptica, Cyclotella meneghiniana, Cyclotella sp.,
Dunaliella sp., Dunaliella bardawil, Dunaliella bioculata, Dunaliella granulate, Dunaliella
maritime, Dunaliella minuta, Dunaliella parva, Dunaliella peircei, Dunaliella primolecta,
Dunaliella salina, Dunaliella terricola, Dunaliella tertiolecta, Dunaliella viridis,
Dunaliella tertiolecta, Eremosphaera viridis, Eremosphaera sp., Ellipsoidon sp., Euglena,
Franceia sp., Fragilaria crotonensis, Fragilaria sp., Gleocapsa sp., Gloeothamnion sp.,
Hymenomonas sp., Isochrysis aff. galbana, Isochrysis galbana, Lepocinclis, Micractinium,
Micractinium (UTEX LB 2614), Monoraphidium minutum, Monoraphidium sp.,
Nannochloris sp., Nannochloropsis salina, Nannochloropsis sp., Navicula acceptata,
Navicula biskanterae, Navicula pseudotenelloides, Navicula pelliculosa, Navicula
saprophila, Navicula sp., Neochloris oleabundans, Nephrochloris sp., Nephroselmis sp.,
Nitschia communis, Nitzschia alexandrina, Nitzschia communis, Nitzschia dissipata,
Nitzschia frustulum, Nitzschia hantzschiana, Nitzschia inconspicua, Nitzschia intermedia,
Nitzschia microcephala, Nitzschia pusilla, Nitzschia pusilla elliptica, Nitzschia pusilla
monoensis, Nitzschia quadrangular, Nitzschia sp., Ochromonas sp., Oocystis parva,
Oocystis pusilla, Oocystis sp., Oscillatoria limnetica, Oscillatoria sp., Oscillatoria
subbrevis, Parachlorella beijerinckii, Parachlorella kessleri, Pascheria acidophila,
Pavlova sp., Phagus, Phormidium, Platymonas sp., Pleurochrysis carterae, Pleurochrysis
dentate, Pleurochrysis sp., Prototheca stagnora, Prototheca portoricensis , Prototheca
moriformis, Prototheca wickerhamii, Prototheca zopfii, Pseudochlorella aquatica,
Pyramimonas sp., Pyrobotrys, Sarcinoid chrysophyte, Scenedesmus armatus , Scenedesmus
rubescens, Schizochytrium, Spirogyra, Spirulina platensis, Stichococcus sp., Synechococcus
sp., Tetraedron, Tetraselmis sp., Tetraselmis suecica, Thalassiosira weissflogii, and
Viridiella fridericiana.

The microorganisms can be genetically engineered to metabolize an alternative sugar source such as sucrose or xylose and/or produce an altered fatty acid profile. Where the microorganism can be grown heterotrophically, it can be an organism that is a permissive or obligate heterotroph. In a specific embodiment, the organism is Prototheca moriformis, an obligate heterotrophic oleaginous microalgae. In a further specific embodiment, the Prototheca moriformis, has been genetically engineered to metabolize sucrose or xylose.

In various embodiments, the microorganism is an organism of a species of the genus Chlorella. In some embodiments, the microalgae is Chlorella protothecoides, Chlorella ellipsoidea, Chlorella minutissima, Chlorella zofinienesi, Chlorella luteoviridis, Chlorella kessleri, Chlorella sorokiniana, Chlorella fusca var. vacuolata Chlorella sp., Chlorella cf. minutissima or Chlorella emersonii. Chlorella is a genus of single-celled green algae, belonging to the phylum Chlorophyta. It is spherical in shape, about 2 to 10 μm in diameter, and is without flagella. Some species of Chlorella are naturally heterotrophic.

Chlorella, for example, Chlorella protothecoides, Chlorella minutissima, or Chlorella emersonii, can be genetically engineered to express one or more heterologous genes (“transgenes”). Examples of expression of transgenes in, e.g., Chlorella, can be found in the literature (see for example PCT Patent Publication Nos. 2010/063031, 2010/063032, and 2008/151149; Current Microbiology Vol. 35 (1997), pp. 356-362; Sheng Wu Gong Cheng Xue Bao. 2000 July; 16(4):443-6; Current Microbiology Vol. 38 (1999), pp. 335-341; Appl Microbiol Biotechnol (2006) 72: 197-205; Marine Biotechnology 4, 63-73, 2002; Current Genetics 39:5, 365-370 (2001); Plant Cell Reports 18:9, 778-780, (1999); Biologia Plantarium 42(2): 209-216, (1999); Plant Pathol. J 21(1): 13-20, (2005)), and such references teach various methods and materials for introducing and expressing genes of interest in such organisms. Other lipid-producing microalgae can be engineered as well, including prokaryotic Microalgae (see Kalscheuer et al., Applied Microbiology and Biotechnology, Volume 52, Number 4/October, 1999).

With regard to the alga species recited herein, it is noted that the taxonomy of algal species is in constant flux. Therefore it is possible that genera, species, and strains will change their names as time progresses. Where possible, alternative strain names are provided. However, it is anticipated that the current status of genus and species designations will change over time and the invention will maintain its relevance to the strains whatever their eventual designation. A current example is the renaming of Chlorella protothecoides as Auxenochlorella protothecoides. For the purposes of this disclosure they should be treated as the same organism.

Prototheca is a genus of single-cell microalgae believed to be a non-photosynthetic mutant of Chlorella. While Chlorella can obtain its energy through photosynthesis, species of the genus Prototheca are obligate heterotrophs. Prototheca are spherical in shape, about 2 to 15 micrometers in diameter, and lack flagella. In various embodiments, the microalgae used to generate the triacylglycerides is selected from the following species of Prototheca: Prototheca stagnora, Prototheca portoricensis, Prototheca moriformis, Prototheca wickerhamii and Prototheca zopfii.

In addition to Prototheca and Chlorella, other microalgae can be used to as sources of triacylglycerides. In various preferred embodiments, the microalgae is selected from a genus or species from any of the following genera and species: Parachlorella kessleri, Parachlorella beijerinckii, Neochloris oleabundans, Bracteacoccus grandis, Bracteacoccus cinnabarinas, Bracteococcus aerius, Bracteococcus sp. or Scenedesmus rebescens.

The oils produced according to the above methods in some cases are made using a microalgal host cell. As described above, the microalga can be, without limitation, fall in the classification of Chlorophyta, Trebouxiophyceae, Chlorellales, Chlorellaceae, or Chlorophyceae. It has been found that microalgae of Trebouxiophyceae can be distinguished from vegetable oils based on their sterol profiles. Oil produced by Chlorella protothecoides was found to produce sterols that appeared to be brassicasterol, ergosterol, campesterol, stigmasterol, and β-sitosterol, when detected by GC-MS. However, it is believed that all sterols produced by Chlorella have C24β stereochemistry. Thus, it is believed that the molecules detected as campesterol, stigmasterol, and β-sitosterol, are actually 22,23-dihydrobrassicasterol, proferasterol and clionasterol, respectively. Thus, the oils produced by the microalgae described above can be distinguished from plant oils by the presence of sterols with C24β stereochemistry and the absence of C24α stereochemistry in the sterols present. For example, the oils produced may contain 22,23-dihydrobrassicasterol while lacking campesterol; contain Clionasterol, while lacking in β-sitosterol, and/or contain poriferasterol while lacking stigmasterol. Alternately, or in addition, the oils may contain significant amounts of Δ⁷-poriferasterol.

In other embodiments, the oils provided herein are not vegetable oils. Vegetable oils are oils extracted from plants and plant seeds. Vegetable oils can be distinguished from the non-plant oils provided herein on the basis of their oil content. A variety of methods for analyzing the oil content can be employed to determine the source of the oil or whether adulteration of an oil provided herein with an oil of a different (e.g. plant) origin has occurred. The determination can be made on the basis of one or a combination of the analytical methods. These tests include but are not limited to analysis of one or more of free fatty acids, fatty acid profile, total triacylglycerol content, diacylglycerol content, peroxide values, spectroscopic properties (e.g. UV absorption), sterol profile, sterol degradation products, antioxidants (e.g. tocopherols), pigments (e.g. chlorophyll), d13C values and sensory analysis (e.g. taste, odor, and mouth feel). Many such tests have been standardized for commercial oils such as the Codex Alimentarius standards for edible fats and oils.

In some embodiments, the oil content of an oil provided herein comprises, as a percentage of total sterols, less than 20%, 15%, 10%, 5%, 4%, 3%, 2%, or 1% 24-ethylcholest-5-en-3-ol. In some embodiments, the 24-ethylcholest-5-en-3-ol is clionasterol. In some embodiments, the oil content of an oil provided herein comprises, as a percentage of total sterols, at least 1%, 2%, 3%, 4%, 5%, 6%, 7%, 8%, 9%, or 10% clionasterol.

In some embodiments, the oil content of an oil provided herein contains, as a percentage of total sterols, less than 20%, 15%, 10%, 5%, 4%, 3%, 2%, or 1% 24-methylcholest-5-en-3-ol. In some embodiments, the 24-methylcholest-5-en-3-ol is 22,23-dihydrobrassicasterol. In some embodiments, the oil content of an oil provided herein comprises, as a percentage of total sterols, at least 1%, 2%, 3%, 4%, 5%, 6%, 7%, 8%, 9%, or 10% 22,23-dihydrobrassicasterol.

In some embodiments, the oil content of an oil provided herein contains, as a percentage of total sterols, less than 20%, 15%, 10%, 5%, 4%, 3%, 2%, or 1% 5,22-cholestadien-24-ethyl-3-ol. In some embodiments, the 5,22-cholestadien-24-ethyl-3-ol is poriferasterol. In some embodiments, the oil content of an oil provided herein comprises, as a percentage of total sterols, at least 1%, 2%, 3%, 4%, 5%, 6%, 7%, 8%, 9%, or 10% poriferasterol.

In some embodiments, the oil content of an oil provided herein contains ergosterol or brassicasterol or a combination of the two. In some embodiments, the oil content contains, as a percentage of total sterols, at least 5%, 10%, 20%, 25%, 35%, 40%, 45%, 50%, 55%, 60%, or 65% ergosterol. In some embodiments, the oil content contains, as a percentage of total sterols, at least 25% ergosterol. In some embodiments, the oil content contains, as a percentage of total sterols, at least 40% ergosterol. In some embodiments, the oil content contains, as a percentage of total sterols, at least 5%, 10%, 20%, 25%, 35%, 40%, 45%, 50%, 55%, 60%, or 65% of a combination of ergosterol and brassicasterol.

In some embodiments, the oil content contains, as a percentage of total sterols, at least 1%, 2%, 3%, 4% or 5% brassicasterol. In some embodiments, the oil content contains, as a percentage of total sterols less than 10%, 9%, 8%, 7%, 6%, or 5% brassicasterol.

In some embodiments the ratio of ergosterol to brassicasterol is at least 5:1, 10:1, 15:1, or 20:1.

In some embodiments, the oil content contains, as a percentage of total sterols, at least 5%, 10%, 20%, 25%, 35%, 40%, 45%, 50%, 55%, 60%, or 65% ergosterol and less than 20%, 15%, 10%, 5%, 4%, 3%, 2%, or 1% β-sitosterol. In some embodiments, the oil content contains, as a percentage of total sterols, at least 25% ergosterol and less than 5% β-sitosterol. In some embodiments, the oil content further comprises brassicasterol.

Sterols contain from 27 to 29 carbon atoms (C27 to C29) and are found in all eukaryotes. Animals exclusively make C27 sterols as they lack the ability to further modify the C27 sterols to produce C28 and C29 sterols. Plants however are able to synthesize C28 and C29 sterols, and C28/C29 plant sterols are often referred to as phytosterols. The sterol profile of a given plant is high in C29 sterols, and the primary sterols in plants are typically the C29 sterols β-sitosterol and stigmasterol. In contrast, the sterol profile of non-plant organisms contain greater percentages of C27 and C28 sterols. For example the sterols in fungi and in many microalgae are principally C28 sterols. The sterol profile and particularly the striking predominance of C29 sterols over C28 sterols in plants has been exploited for determining the proportion of plant and marine matter in soil samples (Huang, Wen-Yen, Meinschein W. G., “Sterols as ecological indicators”; Geochimica et Cosmochimia Acta. Vol 43. pp 739-745).

In some embodiments the primary sterols in the microalgal oils provided herein are sterols other than β-sitosterol and stigmasterol. In some embodiments of the microalgal oils, C29 sterols make up less than 50%, 40%, 30%, 20%, 10%, or 5% by weight of the total sterol content.

In some embodiments the microalgal oils provided herein contain C28 sterols in excess of C29 sterols. In some embodiments of the microalgal oils, C28 sterols make up greater than 50%, 60%, 70%, 80%, 90%, or 95% by weight of the total sterol content. In some embodiments the C28 sterol is ergosterol. In some embodiments the C28 sterol is brassicasterol.

In addition to microalgae, oleaginous yeast can accumulate more than 20% of their dry cell weight as lipid and so are useful sources of triglycerides. Examples of oleaginous yeast include, but are not limited to, the oleaginous yeast listed in Table 2.

TABLE 2
Oleaginous Yeast.

Candida apicola, Candida sp., Cryptococcus curvatus, Cryptococcus terricolus,
Debaromyces hansenii, Endomycopsis vernalis, Geotrichum carabidarum, Geotrichum
cucujoidarum, Geotrichum histeridarum, Geotrichum silvicola, Geotrichum vulgare,
Hyphopichia burtonii, Lipomyces lipofer, Lypomyces orentalis, Lipomyces starkeyi,
Lipomyces tetrasporous, Pichia mexicana, Rodosporidium sphaerocarpum,
Rhodosporidium toruloides, Rhodotorula aurantiaca, Rhodotorula dairenensis,
Rhodotorula diffluens, Rhodotorula glutinus, Rhodotorula glutinis var. glutinis,
Rhodotorula gracilis, Rhodotorula graminis Rhodotorula minuta, Rhodotorula
mucilaginosa, Rhodotorula mucilaginosa var. mucilaginosa, Rhodotorula terpenoidalis,
Rhodotorula toruloides, Sporobolomyces alborubescens, Starmerella bombicola,
Torulaspora delbruekii, Torulaspora pretoriensis, Trichosporon behrend, Trichosporon
brassicae, Trichosporon domesticum, Trichosporon laibachii, Trichosporon loubieri,
Trichosporon loubieri var. loubieri, Trichosporon montevideense, Trichosporon
pullulans, Trichosporon sp., Wickerhamomyces Canadensis, Yarrowia lipolytica, and
Zygoascus meyerae.

Examples of oleaginous microbes include fungi such as the fungi are listed in Table 3.

TABLE 3
Oleaginous Fungi.

Mortierella, Mortierrla vinacea, Mortierella alpine, Pythium debaryanum, Mucor
circinelloides, Aspergillus ochraceus, Aspergillus terreus, Pennicillium iilacinum,
Hensenulo, Chaetomium, Cladosporium, Malbranchea, Rhizopus, and Pythium

In one embodiment, the microorganism used for the production of triacylglycerides for use in drilling fluids provided herein is a fungus. Examples of suitable fungi (e.g., Mortierella alpine, Mucor circinelloides, and Aspergillus ochraceus) include those that have been shown to be amenable to genetic manipulation, as described in the literature (see, for example, Microbiology, July; 153(Pt.7): 2013-25 (2007); Mol Genet Genomics, June; 271(5): 595-602 (2004); Curr Genet, March; 21(3):215-23 (1992); Current Microbiology, 30(2):83-86 (1995); Sakuradani, NISR Research Grant, “Studies of Metabolic Engineering of Useful Lipid-producing Microorganisms” (2004); and PCT/JP2004/012021).

In other embodiments, a microorganism producing a triglyceride is an oleaginous bacterium. Oleaginous bacteria are bacteria that can accumulate more than 20% of their dry cell weight as lipid. Species of oleaginous bacteria for use in the present methods include species of the genus Rhodococcus, such as Rhodococcus opacus and Rhodococcus sp. Methods of cultivating oleaginous bacteria, such as Rhodococcus opacus, are known in the art (see Waltermann, et al., (2000) Microbiology, 146: 1143-1149).

The oleaginous microorganisms can be cultured for production of triglycerides. This type of culture is typically first conducted on a small scale and, initially, at least, under conditions in which the starting microorganism can grow. Culture for purposes of hydrocarbon production is preferentially conducted on a large scale and under heterotrophic conditions. Preferably, a fixed carbon source such as glucose or sucrose, for example, is present in excess. The culture can also be exposed to light some or all of the time, if desired or beneficial.

Microalgae and most other oleaginous microbes can be cultured in liquid media. The culture can be contained within a bioreactor. Optionally, the bioreactor does not allow light to enter. Alternatively, microalgae can be cultured in photobioreactors that contain a fixed carbon source and/or carbon dioxide and allow light to strike the cells. For microalgae cells that can utilize light as an energy source, exposure of those cells to light, even in the presence of a fixed carbon source that the cells transport and utilize (i.e., mixotrophic growth), nonetheless accelerates growth compared to culturing those cells in the dark. Culture condition parameters can be manipulated to optimize total oil production, the combination of hydrocarbon species produced, and/or production of a particular hydrocarbon species. In some instances, it is preferable to culture cells in the dark, such as, for example, when using extremely large (40,000 liter and higher) fermentors that do not allow light to strike a significant proportion (or any) of the culture.

Culture medium typically contains components such as a fixed nitrogen source, trace elements, optionally a buffer for pH maintenance, and phosphate. Components in addition to a fixed carbon source, such as acetate or glucose, may include salts such as sodium chloride, particularly for seawater microalgae. Examples of trace elements include zinc, boron, cobalt, copper, manganese, and molybdenum, in, for example, the respective forms of ZnCl₂, H₃BO₃, CoC1₂.6H₂O, CuCl₂.2H₂O, MnCl₂.4H₂O and (NH₄)₆MO₇O₂₄.4H₂O. Other culture parameters can also be manipulated, such as the pH of the culture media, the identity and concentration of trace elements and other media constituents.

For organisms able to grow on a fixed carbon source, the fixed carbon source can be, for example, glucose, fructose, sucrose, galactose, xylose, mannose, rhamnose, N-acetylglucosamine, glycerol, floridoside, glucuronic acid, and/or acetate. The one or more exogenously provided fixed carbon source(s) can be supplied to the culture medium at a concentration of from at least about 50 μM to at least 500 mM, and at various amounts in that range (i.e., 100 μM, 500 μM, 5 mM, 50 mM).

Some microalgae species can grow by utilizing a fixed carbon source, such as glucose or acetate, in the absence of light. Such growth is known as heterotrophic growth. For Chlorella protothecoides, for example, heterotrophic growth can result in high production of biomass and accumulation of high lipid content. Thus, an alternative to photosynthetic growth and propagation of microorganisms is the use of heterotrophic growth and propagation of microorganisms, under conditions in which a fixed carbon source provides energy for growth and lipid accumulation. In some embodiments, the fixed carbon energy source comprises cellulosic material, including depolymerized cellulosic material, a 5-carbon sugar, or a 6-carbon sugar.

Methods for the growth and propagation of Chlorella protothecoides to high oil levels as a percentage of dry weight have been reported (see for example Miao and Wu, J. Biotechnology, 2004, 11:85-93 and Miao and Wu, Biosource Technology (2006) 97:841-846, reporting methods for obtaining 55% oil dry cell weight).

PCT Publication WO2008/151149, incorporated herein by reference, describes preferred growth conditions for microalgae such as Chlorella. Multiple species of Chlorella and multiple strains within a species can be grown in the presence of glycerol. The aforementioned patent application describes culture parameters incorporating the use of glycerol for fermentation of multiple genera of microalgae. Multiple Chlorella species and strains proliferate very well on not only purified reagent-grade glycerol, but also on acidulated and non-acidulated glycerol byproduct from biodiesel transesterification. In some instances, microalgae, such as Chlorella strains, undergo cell division faster in the presence of glycerol than in the presence of glucose. In these instances, two-stage growth processes in which cells are first fed glycerol to increase cell density, and are then fed glucose to accumulate lipids can improve the efficiency with which lipids are produced.

Other feedstocks for culturing microalgae under heterotrophic growth conditions include mixtures of glycerol and glucose, mixtures of glucose and xylose, mixtures of fructose and glucose, sucrose, glucose, fructose, xylose, arabinose, mannose, galactose, acetate, and molasses. Other suitable feedstocks include corn stover, sugar beet pulp, and switchgrass in combination with depolymerization enzymes. In various embodiments, a microbe that can utilize sucrose as a carbon source under heterotrophic culture conditions is used to generate the microbial biomass. PCT Publication Nos. 2012/106560, 2011/150410, 2011/150411, 2010/063032, and 2008/151149 which are herein incorporated by reference describe recombinant organisms, including but not limited to Prototheca and Chlorella microalgae, that have been genetically engineered to utilize sucrose as a carbon source. In various embodiments, these or other organisms capable of utilizing sucrose as a carbon source under heterotrophic conditions are cultured in media in which the sucrose is provided in the form of a crude, sucrose-containing material, including but not limited to, sugar cane juice (e.g., thick cane juice) and sugar beet juice.

For lipid and oil production, cells, including recombinant cells, are typically fermented in large quantities. The culturing may be in large liquid volumes, such as in suspension cultures as an example. Other examples include starting with a small culture of cells which expand into a large biomass in combination with cell growth and propagation as well as lipid (oil) production. Bioreactors or steel fermentors can be used to accommodate large culture volumes. For these fermentations, use of photosynthetic growth conditions may be impossible or at least impractical and inefficient, so heterotrophic growth conditions may be preferred.

Appropriate nutrient sources for culture in a fermentor for heterotrophic growth conditions include raw materials such as one or more of the following: a fixed carbon source such as glucose, corn starch, depolymerized cellulosic material, sucrose, sugar cane, sugar beet, lactose, milk whey, molasses, or the like; a nitrogen source, such as protein, soybean meal, cornsteep liquor, ammonia (pure or in salt form), nitrate or nitrate salt; and a phosphorus source, such as phosphate salts. Additionally, a fermentor for heterotrophic growth conditions allows for the control of culture conditions such as temperature, pH, oxygen tension, and carbon dioxide levels. Optionally, gaseous components, like oxygen or nitrogen, can be bubbled through a liquid culture. Other starch (glucose) sources include wheat, potato, rice, and sorghum. Other carbon sources include process streams such as technical grade glycerol, black liquor, and organic acids such as acetate, and molasses. Carbon sources can also be provided as a mixture, such as a mixture of sucrose and depolymerized sugar beet pulp.

A fermentor for heterotrophic growth conditions can be used to allow cells to undergo the various phases of their physiological cycle. As an example, an inoculum of lipid-producing cells can be introduced into a medium followed by a lag period (lag phase) before the cells begin to propagate. Following the lag period, the propagation rate increases steadily and enters the log, or exponential, phase. The exponential phase is in turn followed by a slowing of propagation due to decreases in nutrients such as nitrogen, increases in toxic substances, and quorum sensing mechanisms. After this slowing, propagation stops, and the cells enter a stationary phase or steady growth state, depending on the particular environment provided to the cells.

In one heterotrophic culture method, microorganisms are cultured using depolymerized cellulosic biomass as a feedstock. As opposed to other feedstocks that can be used to culture microorganisms, such as corn starch or sucrose from sugar cane or sugar beets, cellulosic biomass (depolymerized or otherwise) is not suitable for human consumption. Cellulosic biomass (e.g., stover, such as corn stover) is inexpensive and readily available.

Suitable cellulosic materials include residues from herbaceous and woody energy crops, as well as agricultural crops, i.e., the plant parts, primarily stalks and leaves typically not removed from the fields with the primary food or fiber product. Examples include agricultural wastes such as sugarcane bagasse, rice hulls, corn fiber (including stalks, leaves, husks, and cobs), wheat straw, rice straw, sugar beet pulp, citrus pulp, citrus peels; forestry wastes such as hardwood and softwood thinnings, and hardwood and softwood residues from timber operations; wood wastes such as saw mill wastes (wood chips, sawdust) and pulp mill waste; urban wastes such as paper fractions of municipal solid waste, urban wood waste and urban green waste such as municipal grass clippings; and wood construction waste. Additional cellulosics include dedicated cellulosic crops such as switchgrass, hybrid poplar wood, and miscanthus, fiber cane, and fiber sorghum. Five-carbon sugars that are produced from such materials include xylose.

Some microbes are able to process cellulosic material and directly utilize cellulosic materials as a carbon source. However, cellulosic material may need to be treated to increase the accessible surface area or for the cellulose to be first broken down as a preparation for microbial utilization as a carbon source. PCT Patent Publication Nos. 2010/120939, 2010/063032, 2010/063031, and PCT 2008/151149, incorporated herein by reference, describe various methods for treating cellulose to render it suitable for use as a carbon source in microbial fermentations.

Bioreactors can be employed for heterotrophic growth and propagation methods. As will be appreciated, provisions made to make light available to the cells in photosynthetic growth methods are unnecessary when using a fixed-carbon source in the heterotrophic growth and propagation methods described herein.

In certain embodiments, the oleaginous microbe is cultured mixotrophically. Mixotrophic growth involves the use of both light and fixed carbon source(s) as energy sources for cultivating cells. Mixotrophic growth can be conducted in a photobioreactor. Microalgae can be grown and maintained in closed photobioreactors made of different types of transparent or semitransparent material. Such material can include Plexiglass® enclosures, glass enclosures, bags made from substances such as polyethylene, transparent or semi-transparent pipes and other material. Microalgae can be grown and maintained in open photobioreactors such as raceway ponds, settling ponds and other non-enclosed containers. The following discussion of photobioreactors useful for mixotrophic growth conditions is applicable to photosynthetic growth conditions as well.

Microorganisms useful in accordance with the methods provided herein are found in various locations and environments throughout the world. As a consequence of their isolation from other species and their resulting evolutionary divergence, the particular growth medium for optimal growth and generation of oil and/or lipid from any particular species of microbe may need to be experimentally determined. In some cases, certain strains of microorganisms may be unable to grow on a particular growth medium because of the presence of some inhibitory component or the absence of some essential nutritional requirement required by the particular strain of microorganism. There are a variety of methods known in the art for culturing a wide variety of species of microalgae to accumulate high levels of lipid as a percentage of dry cell weight, and methods for determining optimal growth conditions for any species of interest are also known in the art.

Solid and liquid growth media are generally available from a wide variety of sources, and instructions for the preparation of particular media that is suitable for a wide variety of strains of microorganisms can be found, for example, online at utex.org/, a site maintained by the University of Texas at Austin for its culture collection of algae (UTEX). For example, various fresh water and salt water media include those shown in Table 4.

TABLE 4
Algal Media.

| Fresh Water Media | Salt Water Media |
|---|---|
| ½ CHEV Diatom Medium | 1% F/2 |
| ⅓ CHEV Diatom Medium | ½ Enriched Seawater Medium |
| ⅕ CHEV Diatom Medium | ½ Erdschreiber Medium |
| 1:1 DYIII/PEA + Gr+ | ½ Soil + Seawater Medium |
| ⅔ CHEV Diatom Medium | ⅓ Soil + Seawater Medium |
| 2X CHEV Diatom Medium | ¼ ERD |
| Ag Diatom Medium | ¼ Soil + Seawater Medium |
| Allen Medium | ⅕ Soil + Seawater Medium |
| BG11-1 Medium | ⅔ Enriched Seawater Medium |
| Bold 1NV Medium | 20% Allen + 80% ERD |
| Bold 3N Medium | 2X Erdschreiber's Medium |
| Botryococcus Medium | 2X Soil + Seawater Medium |
| Bristol Medium | 5% F/2 Medium |
| CHEV Diatom Medium | 5/3 Soil + Seawater Agar Medium |
| Chu's Medium | Artificial Seawater Medium |
| CR1 Diatom Medium | BG11-1 + .36% NaCl Medium |
| CR1+ Diatom Medium | BG11-1 + 1% NaCl Medium |
| CR1-S Diatom Medium | Bold 1NV:Erdshreiber (1:1) |
| Cyanidium Medium | Bold 1NV:Erdshreiber (4:1) |
| Cyanophycean Medium | Bristol-NaCl Medium |
| Desmid Medium | Dasycladales Seawater Medium |
| DYIII Medium | Enriched Seawater Medium |
| Euglena Medium | Erdschreiber's Medium |
| HEPES Medium | ES/10 Enriched Seawater Medium |
| J Medium | ES/2 Enriched Seawater Medium |
| Malt Medium | ES/4 Enriched Seawater Medium |
| MES Medium | F/2 Medium |
| Modified Bold 3N Medium | F/2 + NH4 |
| Modified COMBO Medium | LDM Medium |
| N/20 Medium | Modified 2 X CHEV |
| Ochromonas Medium | Modified 2 X CHEV + Soil |
| P49 Medium | Modified Artificial Seawater Medium |
| Polytomella Medium | Modified CHEV |
| Proteose Medium | Porphridium Medium |
| Snow Algae Media | Soil + Seawater Medium |
| Soil Extract Medium | SS Diatom Medium |
| Soilwater: BAR Medium | |
| Soilwater: GR− Medium | |
| Soilwater: GR−/NH4 Medium | |
| Soilwater: GR+ Medium | |
| Soilwater: GR+/NH4 Medium | |
| Soilwater: PEA Medium | |
| Soilwater: Peat Medium | |
| Soilwater: VT Medium | |
| Spirulina Medium | |
| Tap Medium | |
| Trebouxia Medium | |
| Volvocacean Medium | |
| Volvocacean-3N Medium | |
| Volvox Medium | |
| Volvox-Dextrose Medium | |
| Waris Medium | |
| Waris + Soil Extract Medium | |

A medium suitable for culturing Chlorella protothecoides comprises Proteose Medium. This medium is suitable for axenic cultures, and a 1 L volume of the medium (pH ˜6.8) can be prepared by addition of 1 g of proteose peptone to 1 liter of Bristol Medium. Bristol medium comprises 2.94 mM NaNO₃, 0.17 mM CaCl₂.2H₂O, 0.3 mM MgSO₄.7H₂O, 0.43 mM, 1.29 mM KH₂PO₄, and 1.43 mM NaCl in an aqueous solution. For 1.5% agar medium, 15 g of agar can be added to 1 L of the solution. The solution is covered and autoclaved, and then stored at a refrigerated temperature prior to use.

Other suitable media for use with the methods provided herein can be readily identified by consulting the URL identified above, or by consulting other organizations that maintain cultures of microorganisms, SAG the Culture Collection of Algae at the University of Gottingen (Göttingen, Germany), CCAP the culture collection of algae and protozoa managed by the Scottish Association for Marine Science (Scotland, United Kingdom), and CCALA the culture collection of algal laboratory at the Institute of Botany (Tr\check{e}bo\check{n}, Czech Republic).

Process conditions can be adjusted to increase the percentage weight of cells that is lipid. For example, in certain embodiments, a microbe (e.g., a microalgae) is cultured in the presence of a limiting concentration of one or more nutrients, such as, for example, nitrogen and/or phosphorous and/or sulfur, while providing an excess of fixed carbon energy such as glucose. Nitrogen limitation tends to increase microbial lipid yield over microbial lipid yield in a culture in which nitrogen is provided in excess. In particular embodiments, the increase in lipid yield is from at least about 10% to 100% to as much as 500% or more. The microbe can be cultured in the presence of a limiting amount of a nutrient for a portion of the total culture period or for the entire period. In particular embodiments, the nutrient concentration is cycled between a limiting concentration and a non-limiting concentration at least twice during the total culture period. In one embodiment, the C10-C14 content of the microbial biomass used in the methods comprises at least about 10%, at least about 20%, at least about 30%, at least about 40%, at least about 50%, or at least about 60%, or at least 70% of the lipid content of the biomass. In another aspect, the saturated lipid content of the microbial biomass is at least about 50%, at least about 60%, at least about 70%, at least about 80%, or at least about 90% of the lipid of the microbial biomass.

To increase lipid as a percentage of dry cell weight, acetate can be employed in the feedstock for a lipid-producing microbe (e.g., a microalgae). Acetate feeds directly into the point of metabolism that initiates fatty acid synthesis (i.e., acetyl-CoA); thus providing acetate in the culture can increase fatty acid production. Generally, the microbe is cultured in the presence of a sufficient amount of acetate to increase microbial lipid yield, and/or microbial fatty acid yield, specifically, over microbial lipid (e.g., fatty acid) yield in the absence of acetate. Acetate feeding is a useful component of the methods provided herein for generating microalgal biomass that has a high percentage of dry cell weight as lipid.

In a steady growth state, the cells accumulate oil (lipid) but do not undergo cell division. In one embodiment, the growth state is maintained by continuing to provide all components of the original growth media to the cells with the exception of a fixed nitrogen source. Cultivating microalgae cells by feeding all nutrients originally provided to the cells except a fixed nitrogen source, such as through feeding the cells for an extended period of time, can result in a high percentage of dry cell weight being lipid. In some embodiments, the nutrients, such as trace metals, phosphates, and other components, other than a fixed carbon source, can be provided at a much lower concentration than originally provided in the starting fermentation to avoid “overfeeding” the cells with nutrients that will not be used by the cells, thus reducing costs.

In other embodiments, high lipid (oil) biomass can be generated by feeding a fixed carbon source to the cells after all fixed nitrogen has been consumed for extended periods of time, such as from at least 8 to 16 or more days. In some embodiments, cells are allowed to accumulate oil in the presence of a fixed carbon source and in the absence of a fixed nitrogen source for over 30 days. Preferably, microorganisms grown using conditions described herein and known in the art comprise lipid in a range of from at least about 10% lipid by dry cell weight to about 75% lipid by dry cell weight. Such oil rich biomass can be used directly as a fluid loss control agent in drilling fluids, but often, the spent biomass remaining after lipid has been extracted from the microbes will be used as the fluid loss control agent.

Another tool for allowing cells to accumulate a high percentage of dry cell weight as lipid involves feedstock selection. Multiple species of Chlorella and multiple strains within a species of Chlorella accumulate a higher percentage of dry cell weight as lipid when cultured in the presence of biodiesel glycerol byproduct than when cultured in the presence of equivalent concentrations of pure reagent grade glycerol. Similarly, Chlorella can accumulate a higher percentage of dry cell weight as lipid when cultured in the presence of an equal concentration (weight percent) mixture of glycerol and glucose than when cultured in the presence of only glucose.

Another tool for allowing cells to accumulate a high percentage of dry cell weight as lipid involves feedstock selection as well as the timing of addition of certain feedstocks. For example, Chlorella can accumulate a higher percentage of dry cell weight as lipid when glycerol is added to a culture for a first period of time, followed by addition of glucose and continued culturing for a second period of time, than when the same quantities of glycerol and glucose are added together at the beginning of the fermentation. See PCT Publication No. 2008/151149, incorporated herein by reference.

Triglycerides can be isolated from oleaginous microbes by mechanical pressing with pressure sufficient to extract oil. In various embodiments, the pressing step will involve subjecting the oleaginous microbes to at least 10,000 psi of pressure. In various embodiments, the pressing step involves the application of pressure for a first period of time and then application of a higher pressure for a second period of time. This process may be repeated one or more times (“oscillating pressure”). In various embodiments, moisture content of the oleaginous microbes is controlled during the pressing step. In various embodiments, the moisture is controlled in a range of from 0.1% to 3% by weight.

Expeller presses (screw presses) are routinely used for mechanical extraction of oil from soybeans and oil seeds. Generally, the main sections of an expeller press include an intake, a rotating feeder screw, a cage or barrel, a worm shaft and an oil pan. The expeller press is a continuous cage press, in which pressure is developed by a continuously rotating worm shaft. An extremely high pressure, approximately 10,000-20,000 pounds per square inch, is built up in the cage or barrel through the action of the worm working against an adjustable choke, which constricts the discharge of the pressed cake (spent biomass) from the end of the barrel. In various embodiments, screw presses from the following manufacturers are suitable for use: Anderson International Corp. (Cleveland, Ohio), Alloco (Santa Fe, Argentina), De Smet Rosedowns (Humberside, UK), The Dupps Co. (Germantown, Ohio), Grupo Tecnal (Sao Paulo, Brazil), Insta Pro (Des Moines, Iowa), French Oil Mill (Piqua, Ohio), Harburg Freudenberger (previously Krupp Extraktionstechnik) (Hamburg, Germany), Maschinenfabrik Reinartz (Neuss, Germany), Shann Consulting (New South Wales, Australia) and SKET (Magdeburg, Germany).

### Drilling, Production, and Pumping-Services Fluids

Due to the protection afforded by encapsulation, the encapsulated oils provided herein can be proactively added to drilling fluid systems (e.g. water-based systems), where it circulates through the system until conditions are met to break the encapsulation and release the oil lubricant.

The fluids provided herein include aqueous and non-aqueous drilling fluids and other well-related fluids including those used for production of oil or natural gas, for completion operations, sand control operations, workover operations, and for pumping-services such as cementing, hydraulic fracturing, and acidification. In one embodiment, a fluid includes a fluid loss control agent that is biomass from an oleaginous microbe. In one embodiment, the biomass comprises intact, lysed or partly lysed cells with greater than 5%, 10%, 20%, 30%, 40%, 50%, 60%, 70%, 80%, or 90% oil. In another embodiment, the biomass is spent biomass from which oil has been removed. For example, the oil may be removed by a process of drying and pressing and optionally solvent-extracting with hexane or other suitable solvent. In a specific embodiment, the biomass is dried to less than 6% moisture by weight, followed by application of pressure to release more than 25% of the lipid. Alternately, the cells may be intact, which, when used in a drilling fluid, may impart improved fluid-loss control in certain circumstances. Generally, the drilling fluid can contain about 0.1% to about 20% by weight of said biomass, but in various embodiments, this amount may range from about 0.1% to about 10% by weight of said biomass; from about 0.1% to about 5% by weight of said biomass; from about 0.5% to about 4% by weight of said biomass; and from about 1% to about 4% by weight of said biomass.

In various embodiments, the fluid comprises a fluid loss control agent that is not derived from oleaginous microbial biomass. Suitable fluid loss control agents may include, but are not limited to, unmodified starch, hydroxypropl starch, carboxymethyl starch, unmodified cellulose, carboxymethylcellulose, hydroxyethyl cellulose, and polyanionic cellulose.

The fluid can include an aqueous or non-aqueous solvent. The fluid can also optionally include one or more additional components so that the fluid is operable as a drilling fluid, a drill-in fluid, a workover fluid, a spotting fluid, a cementing fluid, a reservoir fluid, a production fluid, a fracturing fluid, or a completion fluid.

In various embodiments, the fluid is a drilling fluid and the added biomass from the oleaginous microbe serves to help transport cuttings, lubricate and protect the drill bit, support the walls of the well bore, deliver hydraulic energy to the formation beneath the bit, and/or to suspend cuttings in the annulus when drilling is stopped.

When used in a drilling fluid, the biomass may operate to occlude pores in the formation, and to form or promote the formation of a filter cake.

In various embodiments, the fluid is a production fluid and the biomass serves to inhibit corrosion, separate hydrocarbons from water, inhibit the formation of scale, paraffin, or corrosion (e.g., metal oxides), or to enhance production of oil or natural gas from the well. In an embodiment, the biomass is used to stimulate methanogenesis of microbes in the well. The biomass may provide nutrients and/or bind inhibitors so as to increase production of natural gas in the well. In this embodiment, the well can be a coal seam having methane generating capacity. See, for example, US Patent Application Nos. 2004/0033557, 2012/0021495, 2011/0284215, US2010/0248322, 2010/0248321, 2010/0035309, and 2007/0248531.

In various embodiments, the fluid comprises a viscosifier. Suitable viscosifiers include, but are not limited to, an alginate polymer selected from the group consisting of sodium alginate, sodium calcium alginate, ammonium calcium alginate, ammonium alginate, potassium alginate, propyleneglycol alginate, and mixtures thereof. Other suitable viscosifiers include organophillic clay, polyacrylamide, xanthan gum, and mixtures of xanthan gum and a cellulose derivative, including those wherein the weight ratio of xanthan gum to cellulose derivative is in the range from about 80:20 to about 20:80, and wherein the cellulose derivative is selected from the group consisting of hydroxyethylcellulose, hydroxypropylcellulose, carboxymethylcellulose and mixtures thereof. Other suitable viscosifiers include a biopolymer produced by the action of bacteria, fungi, or other microorganisms on a suitable substrate.

Mixtures of a bentonitic clay and additives can also be used as viscosifiers. The additives used in such mixtures can comprise, for example: (a) a nonionic, water-soluble polysaccharide selected from the group consisting of a non-ionic, water-soluble cellulosic derivative and a non-ionic water-soluble guar derivative; (b) an anionic water-soluble polysaccharide selected from the group consisting of a carboxymethyl cellulose and Xanthomonas campestris polysaccharide or a combination thereof; (c) an intermediate molecular weight polyglycol, i.e., selected from the group consisting of polyethylene glycol, polypropylene glycol, and poly-(alkanediol), having an average molecular weight of from about 600 to about 30,000; and (5) compatible mixtures thereof. Components of the mixtures may be added individually to the fluid to enhance the low shear rate viscosity thereof.

In some embodiments, the drilling fluid comprises one or more additives selected from the group consisting of an aphron, polymer particle, a thermoset polymer particle, and a nanocomposite particulate.

Aphrons can be used as additives to drilling fluids and other fluids used in creating or maintaining a borehole. Aphrons can concentrate at the fluid front and act as a fluid loss control agent and/or bridging agent to build an internal seal of the pore network along the side walls of a borehole. It is believed that aphrons deform during the process of sealing the pores and gaps encountered while drilling a borehole. Aphrons useful in the present methods are typically 50-100 μM, 25-100 μM, 25-50 μM, 5-50, 5-25 μM, 7-15 μM or about 10 μM.

In one embodiment, a drilling fluid comprises aphrons, microbial biomass in which the oil has not been extracted (unextracted microbial biomass), spent biomass or a combination of aphrons, unextracted microbial biomass, and spent biomass.

Where an aphron is used, the aphron can have an average diameter of 5 to 50 micrometers and can make up about 0.001% to 5% by mass of the fluid.

The use of drilling fluids containing polymer particle additives has several applications in construction, drilling, completion, and fracture simulation of oil and natural gas wells. These particles are generally spherical in shape, solid, and have a specific gravity of 1.06. The use of these particles provides several advantages, such as increasing mechanical lubrication, reducing equipment wear, and aiding in directional changes during sliding. These particles are generally resistant to deformation loads of up to >25,000 psi hydrostatic, and they display excellent resistance and thermal stability even at temperatures greater than 450° F. in a drilling environment. These particles can also be manufactured in fine or coarse grades, depending on the requirements of a particular drilling operation.

Polymer particles are easily added to drilling fluid through a mud-mixing hopper machine. When used to control torque and drag, these beads can be applied at concentrations of 2-8 ppb (5.71-22.87 kilograms/m³). For spotting in wire-line operations and running casing, the polymer beads may be added to concentrations of 8-12 ppb (22.87-34.31 kilograms/m³).

In some embodiments, the drilling fluid comprises a thermoset polymer particle such as those disclosed in U.S. Pat. No. 8,088,718. In some embodiments, the drilling fluid comprises a nanocomposite particulate such as those disclosed in US 2005/0272611. In some embodiments, the drilling fluid comprises a co-polymer bead such as Alpine Drill Beads commercially available from Alpine Specialty Chemicals (Houston, Tex.).

Examples of other additives that may be used in drilling applications include, but are not limited to: alkalinity agents, corrosion inhibitors, defoamers, dispersants, emulsifiers, fluid loss control agents, foaming agent for gas-based fluids, intermediates for corrosion inhibitor, lubricants, misting agents, oxygen scavengers, hydrosulfite scavengers, biocides, scale inhibitors, scale removers, shale inhibitors, solvents, specialty surfactants, thermal stabilizers, viscosifiers, and water purifiers.

The additives disclosed herein, e.g., including the polymeric and glass bead additives, can contribute to bursting and releasing oil from the microbial cells. In such instances the additives work in concert with the cells to provide delay-released lubrication to the drill bit. Though not intended to be limited by the following mechanism, in one aspect this application is directed to a pressure sensitive lubricant that allows for time-delayed release of a lubricating oil by virtue of the oil being encapsulated within a cell. In instances when the lubricant is used in a drilling fluid, the pressure that triggers the oil to be released is provided by the drill string and/or drill bit. The oil is released only when sufficient downhole pressure and/or friction is present. Such pressure and friction is provided by the drill string and/or drill bit in its interaction with the well formation, such as when it is dragged along the well-bore (particularly in the non-vertical portions of the well-bore) or during the rotational motion of the drill string/drill bit during drilling.

Additives and lubricants to be used in combination with the oleaginous cells and oils provided herein include commercially available lubricants. These lubricants can be blended with oleaginous cells and oils produced by these cells. The commercially available lubricants include those marketed by Baker Hughes (RHEO-LOGIC, MAGMA-TEQ, CARBO-DRILL, MPRESS, PERFORMAX, PERFLEX, TERRA-MAX, PYRO-DRILL, MAX-BRIDGE, CHEK-LOSS, LC-LUBE, MIL-CARB, SOLUFLAKE, FLOW-CARB, X-LINK crosslinked composition, and SOLU-SQUEEZE LCM), Haliburton (BAROID, BOREMAX, PERFORMADRIL, SHALEDRIL, SUPER-SAT, and BaraECD) and Schlumberger (DRILPLEX, DURATHERM, ENVIROTHERM NT, GLYDRIL, K-MAG, KLA-SHIELD, SAGDRIL, ULTRADRIL, ECOGREEN, MEGADRIL, NOVAPLUS, PARADRIL, PARALAND, PARATHERM, RHADIANT, VERSACLEAN, VERSADRIL, and WARP fluids).

In various embodiments, the fluid comprises a density modifier, also known as a weighting agent or a weighting additive. Suitable density modifiers include, but are not limited to, barite, hematite, manganese oxide, calcium carbonate, iron carbonate, iron oxide, lead sulfide, siderate, and ilmenite.

In various embodiments, the fluid comprises an emulsifier. Suitable emulsifiers may be nonionic, including ethoxylated alkylphenols and ethoxylated linear alcohols, or anionic, including alkylaryl sulfonates, alcohol ether sulfonates, alkyl amine sulfonates, petroleum sulfonates, and phosphate esters.

In various embodiments, the fluid comprises a lubricant. Non-limiting, suitable lubricants may include fatty acids, tall oil, sulphonated detergents, phosphate esters, alkanolamides, asphalt sulfonates, graphite, and glass beads.

The fluid can be a drilling fluid with a low shear rate viscosity as measured with a Brookfield viscometer at 0.5 rpm of at least 20,000 centipoise. In some embodiments, the low shear rate viscosity is at least about 40,000 centipoise.

Biomass added to fluid can be chemically modified prior to use. Chemical modification involves the formation or breaking of covalent bonds. For example, the biomass may be chemically modified by transesterification, saponification, crosslinking or hydrolysis. The biomass may be treated with one or more reactive species so as to attach desired moieties. The moieties may be hydrophobic, hydrophilic, amphiphilic, ionic, or zwitterionic. For example, the biomass may anionized (e.g., carboxymethylated), or acetylated. Methods for covalent modification including carboxymethylation and acetylation of biomass from oleaginous microbes are disclosed in U.S. Provisional Patent Application No. 61/615,832, filed on Mar. 26, 2012 for “Algal Plastics and Absorbants”, incorporated herein by reference in relevant part. U.S. Pat. No. 3,795,670 describes an acetylation process that can be used to increase the hydrophobicity of the biomass by reaction with acetic anhydride. Carboxymethylation of the biomass can be performed by treatment with monochloroacetic acid. See, e.g., U.S. Pat. No. 3,284,441. U.S. Pat. Nos. 2,639,239; 3,723,413; 3,345,358; 4,689,408, 6,765,042, and 7,485,719, which disclose methods for anionizing and/or cross-linking.

The fluid can include one or more additives such as bentonite, xanthan gum, guar gum, starch, carboxymethylcellulose, hydroxyethyl cellulose, polyanionic cellulose, a biocide, a pH adjusting agent, polyacrylamide, an oxygen scavenger, a hydrogen sulfide scavenger, a foamer, a demulsifier, a corrosion inhibitor, a clay control agent, a dispersant, a flocculant, a friction reducer, a bridging agent, a lubricant, a viscosifier, a salt, a surfactant, an acid, a fluid loss control additive, a gas, an emulsifier, a density modifier, diesel fuel, and an aphron.

Fluids may be mixed or sheared for times appropriate to achieve a homogenous mixture.

Fluids may be subject to aging prior to testing or use. Aging may be performed under conditions that vary from static to dynamic and from ambient (20-25° C.) to highly elevated temperatures (>250° C.).

Fluids can be described as Newtonian or non-Newtonian depending on their response to shearing. The shear stress of a Newtonian fluid is proportional to the shear rate. For non-Newtonian fluids, viscosity decreases as shear rate increases. One classification of non-Newtonian fluid behavior, pseudoplastic behavior, refers to a general type of shear-thinning that may be desirable for drilling fluids. Several mathematical models known in that art have been developed to describe the shear stress/shear rate relationship of non-Newtonian fluids. These models, including the Bingham plastic model, the Power Law model, and the Herschel-Buckley Model are described in “The Drilling Fluids Processing Handbook, Shale Shaker Committee of the American Society of Mechanical Engineers eds, Gulf Professional Publishing, 2004”. Additionally, see reference manuals including “Drilling Fluids Reference Manual, 2006” available from Baker Hughes.

### EXAMPLES

### Example 1

Metal to metal lubricity tests were conducted using hot rolled lab formulated drilling fluids. The fluids were prepared by mixing a water-based, synthetic based, or oil based mud with microalgal cells and/or free oil extracted from the cells. The drilling fluids were hot rolled for 16 hours at atmospheric pressure and standard temperatures (150° F. for oil-based mud, 120° F. for water-based mud and synthetic-based mud).

The muds were prepared using the formulations provided in Tables 5-7. Strain A was derived from UTEX 1435 by classical mutagenesis and screened for high oil production. Strain B was also derived from UTEX 1435 by classical mutagenesis and screened for high oil production, and was further transformed according to WO 2010/063031 to express a yeast sucrose invertase. The fatty acid profiles of oil from Strains A and B are given in Table 5.

TABLE 5
Fatty acid profile of oil from Strains A and B

| Fatty acid | Strain A | Strain B |
|---|---|---|
| C10:0 | 0.0 | 0.0 |
| C12:0 | 0.1 | 0.0 |
| C14:0 | 2.3 | 0.7 |
| C16:0 | 27.5 | 13.2 |
| C18:0 | 2.03 | 5.2 |
| C18:1 | 59.0 | 71.8 |
| C18:2 | 6.2 | 6.4 |
| C18:3 | 0.2 | 0.1 |
| C20:0 | 0.2 | 0.4 |

The strains were cultured under heterotrophic conditions such as those described in WO2008/151149, WO2010/063031, WO2010/063032, WO2011/150411, and WO2013/158938.

Upon cultivation, broth from lots corresponding to different fermentation runs were dried using a drum dryer. Resulting solid biomass are shown in Table 6 below, and are identified according to strain (A or B) and, where applicable, lot number (1-4). A2 was prepared by taking the drum dried biomass and resuspending to 20% solids in deionized water, treating twice with a homogenizer at 1250 bar, and freeze drying.

TABLE 6
Biomass

| Biomass | Processing |
|---|---|
| A1 | Drum dried |
| A2 | Drum dried and homogenized (2x) |
| A3 | Drum dried |
| A4 | Drum dried |
| B | Drum dried |

Water based, synthetic based, or salty water based mud containing 3% or 6% by volume of the biomass were prepared as described in Tables 7-10.

TABLE 7
Mud A: Water-based drilling fluid

| Component | Amount | | Comments |
|---|---|---|---|
| Tap Water | 4 | liters | Add to mixing container |
| Sodium Bicarbonate | 5 | grams | Slowly add and mix for 5 minutes |
| Bentonite | 56 | grams | Slowly add and mix for a minimum |
| | | | of 16 hours |
| Low Viscosity CMC | 14 | grams | Slowly add and mix for 5 minutes |
| Xanthum Gum | 14 | grams | Slowly add and mix for 5 minutes |
| Barite | 18 | grams | Slowly add and mix for 30 minutes; |
| | | | hot roll mixture for 16 hours at |
| | | | 120° C. |

TABLE 8
Mud B: Synthetic based drilling fluid

| Component | Amount | | Comments |
|---|---|---|---|
| Escaid 110 | 1698 | grams | Add to mixing container |
| EZ-Mul | 60 | grams | Slowly add and mix for 5 minutes |
| Tap Water | 728 | grams | Slowly add and mix for 5 minutes |
| Calcium chloride | 254 | grams | Slowly add and mix for 5 minutes |
| Lime | 30 | grams | Slowly add and mix for 5 minutes |
| Gel-tone II | 80 | grams | Slowly add and mix for 5 minutes |
| Duratone HT | 60 | grams | Slowly add and mix for 5 minutes |
| RM-63 | 6 | grams | Slowly add and mix for 5 minutes |
| Barite | 1210 | grams | Slowly add and mix for 30 minutes; |
| | | | hot roll mixture for 16 hours at |
| | | | 150° C. |

TABLE 9
Mud C: Salty water-based drilling fluid

| Component | Amount | | Comments |
|---|---|---|---|
| Tap Water | 1383.2 | | Add to mixing container |
| Quick Thin | 50 | grams | Slowly add and mix for 5 minutes |
| Aquagel | 200 | grams | Slowly add and mix for 5 minutes |
| PAC LV | 5 | grams | Slowly add and mix for 5 minutes |
| Seawater salts | 1483 | grams | Slowly add and mix for 5 minutes |
| Carbonox LV-CMC | 30 | grams | Slowly add and mix for 5 minutes |
| Sodium hydroxide | 20 | grams | Slowly add and mix for 5 minutes |
| Soda Ash | 10 | gram | Slowly add and mix for 5 minutes |
| Rev Dust | 500 | grams | Slowly add and mix for 5 minutes |
| Barite | 1785 | grams | Slowly add and mix for 30 |
| | | | minutes; hot roll mixture for 16 |
| | | | hours at 120° C. |

TABLE 10
Mud D: Water-based drilling fluid

| Component | Amount | | Comments |
|---|---|---|---|
| Tap Water | 337.97 | | Add to mixing container |
| Bentonite | 20 | grams | Slowly add and allow bentonite to |
| | | | hydrate for minimum of 16 hours |
| Lignite | 2 | grams | Slowly add and mix for 5 minutes |
| Chrome free | 0.5 | grams | Slowly add and mix for 5 minutes |
| lignosulfonate | | | |
| Polyanionic | 1.5 | grams | Slowly add and mix for 5 minutes |
| cellulose | | | |
| Xanthum gum | 0.75 | grams | Slowly add and mix for 30 minutes; |
| | | | hot roll mixture for 16 hours at |
| | | | 120° C. |

The metal to metal lubricity coefficients (coefficients of friction) were determined using a Fann EP/Lubricity Tester Model 21200. In this test, 150 inch-pounds of force was applied between two hardened steel surfaces, a block and a rotating ring, at 60 RPM with readings taken at the indicated time points in Tables 11-15.

TABLE 11
Metal to metal lubricity with water based drilling mud

| | 1 | 3 | 5 | 10 | 30 | 60 |
|---|---|---|---|---|---|---|
| Sample | Min. | Min. | Min. | Min. | Min. | Min. |
| Mud A | 0.32 | 0.32 | 0.32 | 0.32 | | |
| Mud A + 3% biomass B | 0.27 | 0.24 | 0.25 | 0.26 | 0.25 | |
| Mud A + 3% biomass B | 0.25 | 0.26 | 0.28 | 0.23 | 0.21 | 0.22 |
| Mud A + 3% biomass A1 | 0.24 | 0.24 | 0.24 | 0.24 | 0.23 | |
| Mud A + 3% biomass A2 | 0.24 | 0.22 | 0.22 | 0.20 | 0.14 | 0.13 |
| Mud A + 3% biomass A3 | 0.18 | 0.15 | 0.15 | 0.16 | 0.19 | |
| Mud A + biomass B | 0.08 | 0.06 | 0.06 | 0.05 | 0.04 | 0.04 |
| Mud A + oil from Strain A | 0.04 | 0.04 | 0.04 | 0.04 | 0.05 | 0.05 |
| Mud A + 3% biomass A4 | 0.23 | 0.21 | 0.21 | 0.21 | 0.21 | |
| Mud A + 3% biomass A1 | 0.17 | 0.16 | 0.15 | 0.15 | 0.14 | 0.13 |

TABLE 12
Metal to metal lubricity with water based drilling mud
(measurements taken immediately after hot rolling)

| | 1 | 3 | 5 | 10 | 30 | 60 | 90 |
|---|---|---|---|---|---|---|---|
| Sample | Min. | Min. | Min. | Min. | Min. | Min. | Min. |
| Mud A | 0.32 | 0.32 | 0.32 | 0.32 | | | |
| Mud A + 6% biomass | 0.17 | 0.16 | 0.16 | 0.16 | 0.15 | 0.14 | 0.12 |
| A5 | | | | | | | |
| Mud A + 3% biomass | 0.23 | 0.16 | 0.13 | 0.11 | 0.09 | 0.07 | |
| A5 | | | | | | | |
| Mud A + 3% biomass | 0.20 | 0.15 | 0.15 | 0.13 | 0.11 | 0.09 | |
| A1 | | | | | | | |
| Mud A + 3% biomass | 0.10 | 0.10 | 0.10 | 0.10 | 0.10 | 0.10 | |
| A2 | | | | | | | |
| Mud A + 3% biomass | 0.11 | 0.11 | 0.11 | 0.11 | 0.10 | 0.10 | |
| A3 | | | | | | | |
| Mud A + 3% MIL- | 0.05 | 0.05 | 0.05 | 0.05 | 0.05 | | |
| LUBE | | | | | | | |
| Mud A + 3% Eco | 0.03 | 0.03 | 0.04 | 0.09 | 0.05 | | |
| Global Solutions DFL | | | | | | | |

TABLE 13
Metal to metal lubricity with water based drilling mud

| | 1 | 3 | 5 | 10 | 30 | 60 | 90 |
|---|---|---|---|---|---|---|---|
| Sample | Min. | Min. | Min. | Min. | Min. | Min. | Min. |
| Mud D | 0.24 | 0.26 | 0.27 | 0.27 | 0.27 | 0.26 | |
| Mud D + 3% biomass | 0.23 | 0.23 | 0.23 | 0.23 | 0.21 | 0.19 | |
| B | | | | | | | |
| Mud D + 3% biomass | 0.20 | 0.20 | 0.21 | 0.20 | 0.21 | 0.20 | |
| A2 | | | | | | | |
| Mud D + 3% biomass | 0.13 | 0.13 | 0.13 | 0.13 | 0.13 | 0.14 | 0.14 |
| A2 | | | | | | | |

TABLE 14
Metal to metal lubricity with synthetic based drilling mud

| | 1 | 3 | 5 | 10 | 30 | 60 |
|---|---|---|---|---|---|---|
| Sample | Min. | Min. | Min. | Min. | Min. | Min. |
| Mud B | 0.13 | 0.13 | 0.13 | | | |
| Mud B + 3% biomass A1 | 0.19 | 0.14 | 0.13 | 0.11 | 0.11 | 0.11 |
| Mud B + 3% biomass A2 | 0.10 | 0.09 | 0.10 | 0.10 | 0.10 | 0.10 |
| Mud B + 3% biomass A5 | 0.12 | 0.11 | 0.10 | 0.08 | 0.08 | 0.12 |
| Mud B + oil from Strain A | 0.11 | 0.10 | 0.10 | 0.09 | 0.10 | 0.09 |

TABLE 15
Metal to metal lubricity with salty water based drilling
mud (measurements taken immediately after hot rolling)

| | 1 | 3 | 5 | 10 | 30 | 60 |
|---|---|---|---|---|---|---|
| Sample | Min. | Min. | Min. | Min. | Min. | Min. |
| Mud C | 0.24 | 0.23 | 0.22 | 0.20 | 0.19 | |
| Mud C + oil from Strain A | 0.22 | 0.20 | 0.18 | 0.16 | 0.15 | |
| Mud C + 3% biomass A1 | 0.25 | 0.23 | 0.22 | 0.21 | 0.19 | 0.16 |
| Mud C + 3% biomass A2 | 0.19 | 0.25 | 0.10 | 0.07 | 0.06 | 0.05 |
| Mud C + 3% biomass B | 0.24 | 0.22 | 0.21 | 0.21 | 0.19 | |

The changes in the lubricity of the drilling fluid when the biomass or oils are added can be expressed in Table 16 as a percent reduction in torque (ratio of difference in lubricity to lubricity of mud without microalgal cells/microalgal oil).

TABLE 16
Percent Torque Reduction at 60 minutes

| | | | Salty |
|---|---|---|---|
| | Water-based | Synthetic | water-based |
| Sample | mud | oil -based mud | mud |
| 3% whole cells (Strain A) | 57% | 15% | 13% |
| 3% lysed cells (Strain A) | 58% | 23% | 67% |
| 3% whole cells (Strain B) | 77% | 8% | 2% |

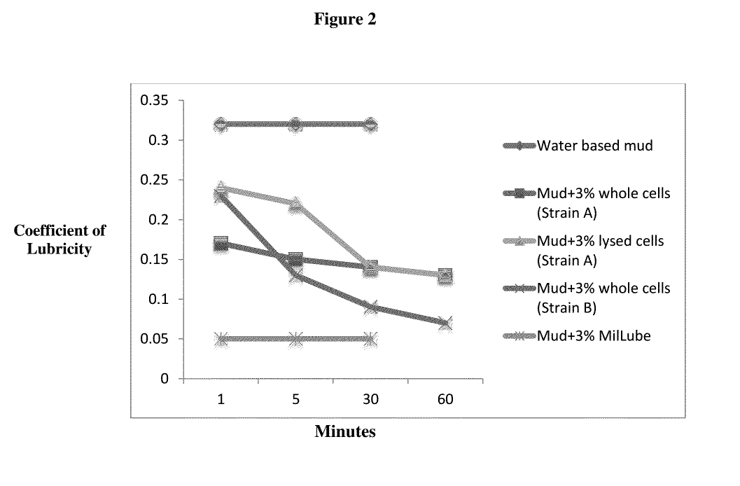

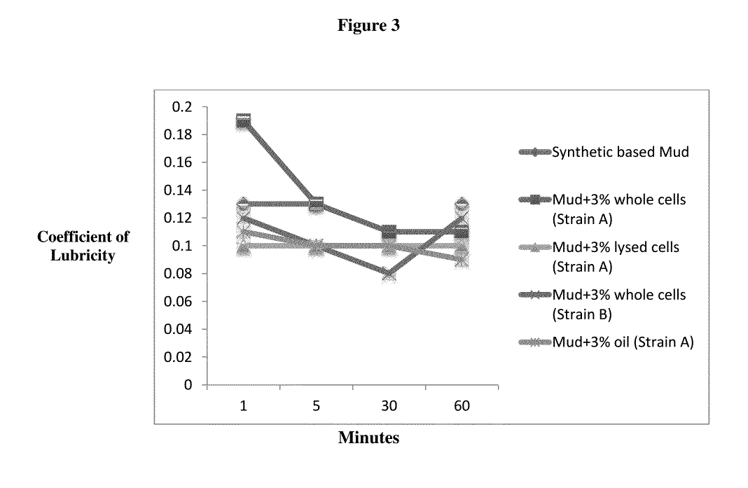

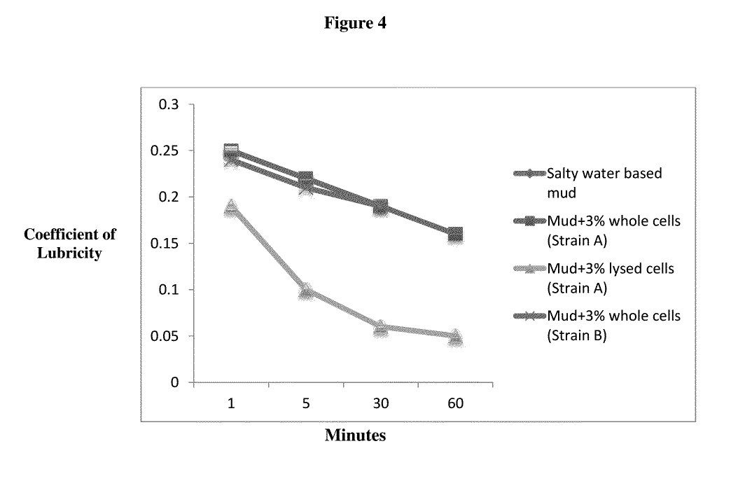

As shown in FIG. 2, the water-based mud formulated with whole or lysed cells demonstrated reduction in coefficient of lubricity as a function of time. Based on the reductions in the coefficient of lubricity, the torque reduction resulting from the use of whole or lysed cells is estimated to be 57-77%. Synthetic based muds containing whole cells were found to demonstrate a trend of decreasing coefficients of lubricity as shown in FIG. 3, corresponding to approximately 8-15% torque reduction. Synthetic based muds containing lysed cells were found to have a lower coefficient of lubricity (0.1), corresponding to a reduction in torque of about 23%. In salty water based muds, formulations with lysed cells showed the greatest decrease in coefficient of lubricity over time, corresponding to a torque reduction of approximately 67% as shown in FIG. 4.

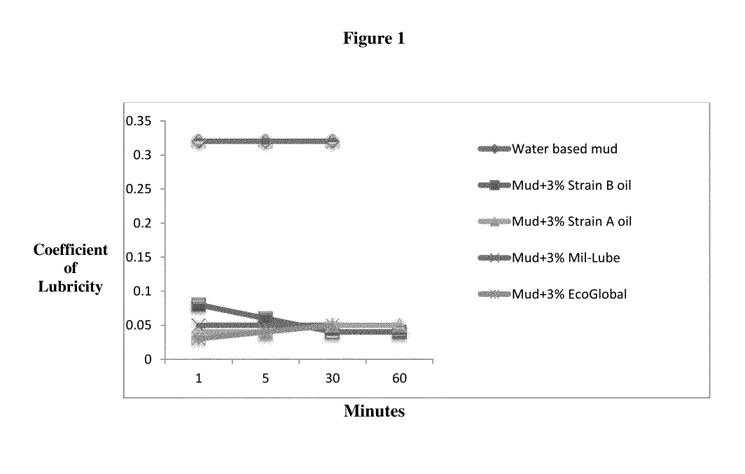

The coefficients of lubricity mud containing the oils from Strains A and B were compared to mud containing commercially available extreme pressure lubricants DFL EcoGlobal or Baker Hughes Mil-Lube, a vegetable oil lube. As seen in FIG. 1 and Table 17, the reductions in coefficient of lubricity and associated torque reduction due to addition of oils from Strain A and B in the mud were found to be comparable to the commercial lubricants.

TABLE 17
Percent Torque Reduction

| | | | Salty |
|---|---|---|---|
| | Water-based | Synthetic | water-based |
| Sample | mud | oil -based mud | mud |
| Free oil (Strain A) | 87% | | |
| Free oil (Strain B) | 84% | 31% | 19% |
| Mil-lube | 84% | | |
| EcoGlobal DFL | 84% | | |

### Example 2

Extreme pressure lubricity tests were performed using Fann EP/Lubricity tester model 21200 with results given in Table 18. Significant increases were seen in film strength upon addition of 3% biomass from strain B.

TABLE 18
Extreme pressure tests

| | | Torque | Scar Width | Film |
|---|---|---|---|---|
| | Sample | (inch | (hundredths of | Strength |
| Sample | Preparation | pounds) | an inch) | (PSI) |
| Mud A | Hot rolled 16 | 150 | 17.50 | 4571 |
| | hrs at 120° C. | | | |
| Mud A + 3% | Hot rolled 16 | 150 | 9.50 | 8421 |
| biomass B | hrs at 120° C. | | | |
| Mud A + 3% oil | Hot rolled 16 | 150 | 15.00 | 5333 |
| from Strain A | hrs at 120° C. | | | |
| Mud B + 3% oil | Hot rolled 16 | 150 | 11.00 | 7273 |
| from Strain A | hrs at 120° C. | | | |
| Mud A + 3% oil | Hot rolled 16 | 150 | 11.50 | 6957 |
| from Strain A | hrs at 120° C. | | | |
| | and blended | | | |
| | 10 minutes | | | |
| | prior to testing | | | |
| Mud A + 3% | Hot rolled 16 | 150 | 7.50 | 10667 |
| MIL-LUBE | hrs at 120° C. | | | |
| | and blended | | | |
| | 10 minutes | | | |
| | prior to testing | | | |
| Mud A + 3% | Hot rolled 16 | 150 | 8.50 | 9412 |
| EcoGlobal DFL | hrs at 120° C. | | | |
| | and blended | | | |
| | 10 minutes | | | |
| | prior to testing | | | |

### Example 3

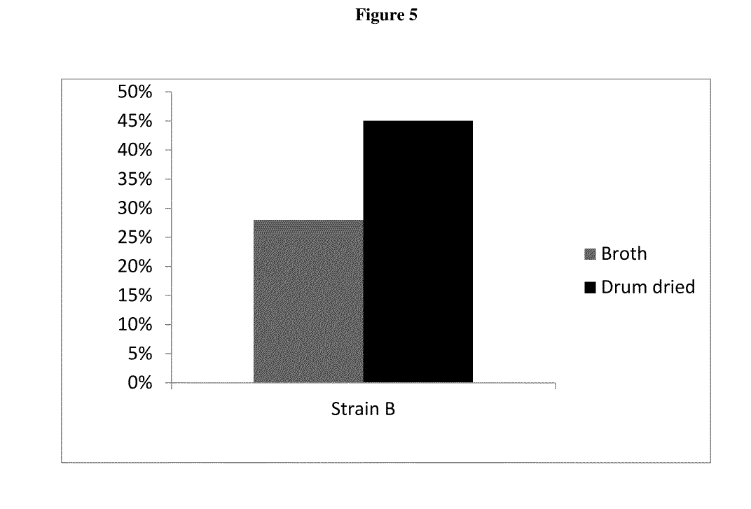

Cells from strain B isolated from the culture broth or drum dried were lysed using a homogenizer at 500 bar pressure (7,252 psi) to determine effect of pressure on cell breakage. As seen Table 19 and FIG. 5, about 45% of the cells were lysed at this pressure, with greater lysis seen in the drum dried biomass.

TABLE 19
Percent lysis at 500 bar

| Strain | Broth | Drum dried |
|---|---|---|
| B | 28 | 45 |

### Example 4

A field trial using water based muds containing microalgal cells from Strain A was conducted to assess efficacy in increasing the rate of penetration and reducing drill bit drag. The water based muds were prepared using a formulation provided in Table 20.

TABLE 20
Water-based drilling mud formulation

| Products | Unit Sizes | Concentrations | | Estimated Usages | |
|---|---|---|---|---|---|
| Xanthan gum | 25 lbs sack | 1.5 | ppb* | 35 | sacks |
| Soda ash | 50 lbs sack | 0.25 | ppb | 3 | sacks |
| White Starch | 50 lbs sack | 4.0 | ppb | 46 | stacks |
| Polyanionic | 50 lbs sack | 0.5 | ppb | 6 | sacks |
| cellulose | | | | | |
| (PAC) LV | | | | | |
| Caustic Soda | 50 lbs sack | 0.15 | ppb | 3 | sacks |
| Glutaraldehyde | 44.6 lbs × 5 gal pail | 0.5 | ppb | 7 | pails |
| Strain A whole | 1 lb | 17 | ppb | 8200 | lbs |
| microalgal cell | | | | | |

*ppb = pounds per barrel

Xanthan gum was used as for rheology control in this trial. Starch is a quality fluid loss additive and was used in the trial to provide excellent low end rheology enhancement in conjunction with xanthan gum. Glutaraldehyde was employed as a biocide. Polyanionic cellulose (PAC) was added for viscosity and filtration control. Caustic soda was added to control alkalinity, while soda ash was used to precipitate hardness to allow calcium-sensitive materials such as PAC to function efficiently. The calcium was controlled between 100-200 ppm with soda ash, and the p_{f} (i.e., a measurement of alkalinity) was controlled between 0.5-1.0 with caustic soda.

Wells having the primary system parameters provided in Tables 21-22 were drilled at a Catoosa Testing Facility in Hallet, Okla., where soil formation at 1300 feet total vertical distance (TVD) was composed of a shale layer.

TABLE 21
System properties

| Properties | Parameters | Units |
|---|---|---|
| Surface Density | 8.6-8.8 | Ppg |
| Low shear rate viscosity | 4,000-8,000 | cPs |
| (LSRV) | | |
| Yield Point (YP) | 8-14 | Lbs/100 ft² |
| 6 θ (contingency hole) | 8-10 | Rpm |
| 3 θ (contingency hole) | 8-10 | Rpm |
| 10 Sec Gel | 6-10 | Lbs/100 ft² |
| 10 Min Gel | 8-14 | Lbs/100 ft² |
| API Fluid Loss (30 min) | >10.0 | cc |

TABLE 22
Drilling parameters

| Hole Size | 8.5 | inches |
|---|---|---|
| Starting Depth: (MD) | 500 | feet |
| Interval TD: (MD) | 1,900 | feet |
| Interval Length | 1,400 | feet |
| Estimated Washout Generated: | 1.0% by volume | |
| Last Casing I.D.: | 9.625 | inches |
| Last Casing Shoe: (MD) | 500 | feet |
| New Surface Volume: | 350 | barrels |
| Volume Carried Forward: | 0 | barrels |
| Open Hole Volume: | 99 | barrels |
| Solids Control Efficiency: | 90.0% | |
| Maximum Drill Solids at Suction: | 5.0% | |
| Flow Rate: | 10 | BPM |
| Maximum Drilled Solids in Annulus: | 8.0% by volume | |
| Volume of Dilution Fluid Used: | 188 | barrels |
| Maximum Uniform Drilling Rate Allowed: | 120 | feet per hour |
| Casing and Open Hole Volume: | 136 | barrels |
| Total Interval Volume: | 575 | barrels |

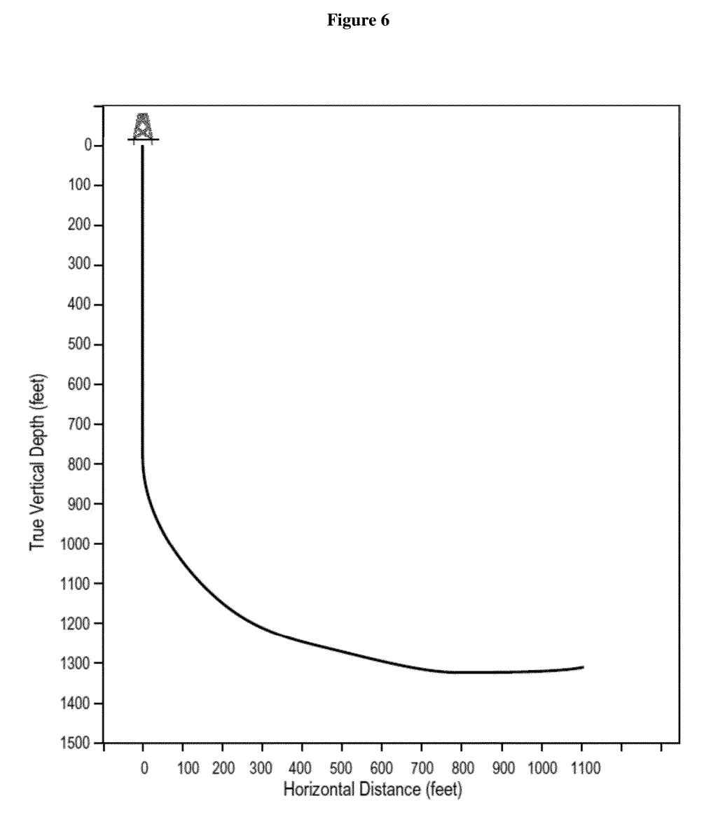

To measure the effect of using whole microalgal cells on the drill bit's rate of penetration (ROP), wells were created by drilling a vertical 8.5 inch diameter hole to the kick off point (KOP) at 750 feet measured depth (MD) and then drilling a curve at 10⁰ per 100 feet, achieving 90⁰ at +/−1650 feet MD, as shown in FIG. 6 (drilled using a 1.5 degree bent-housing motor operated and a GX-30CDX tricone bit (Baker Hughes). After reaching the landing point, 180 feet was drilled laterally by rotating. Whole microalgal cells were then added to the water based mud for the drill bit and allowed to incubate for 1 or 2 hours, before proceeding with drilling along a lateral section. Data was collected on an NOV Totco system connected to a top drive on an oil rig.

The use of the whole cells appeared to increase the rate of penetration by 20% after 2 hour incubation, as shown in Table 23. There was no change in the rate of penetration with the 1 hour incubation period, and this confirmed that circulation time and shearing were necessary to activate lubricity (e.g., weaken cells to enable rupture). This field trial showed that the use of whole cells either reduced drilling time or increased drilling distance.

TABLE 23
Percent Increase in Average Rate of Penetration (ROP)

| | | | % increase in ROP |
|---|---|---|---|
| | Average ROP | Standard | relative to No |
| Sample | (ft/hr) | deviation | MEOCs |
| Mud | 56 | 4 | N/A |
| Mud + whole | 56 | 5 | 0 |
| microalgal cells from | | | |
| Strain A + 1 hr | | | |
| incubation | | | |
| Mud + whole | 68 | 5 | 20% |
| microalgal cells from | | | |
| Strain A + 2 hr | | | |
| incubation | | | |

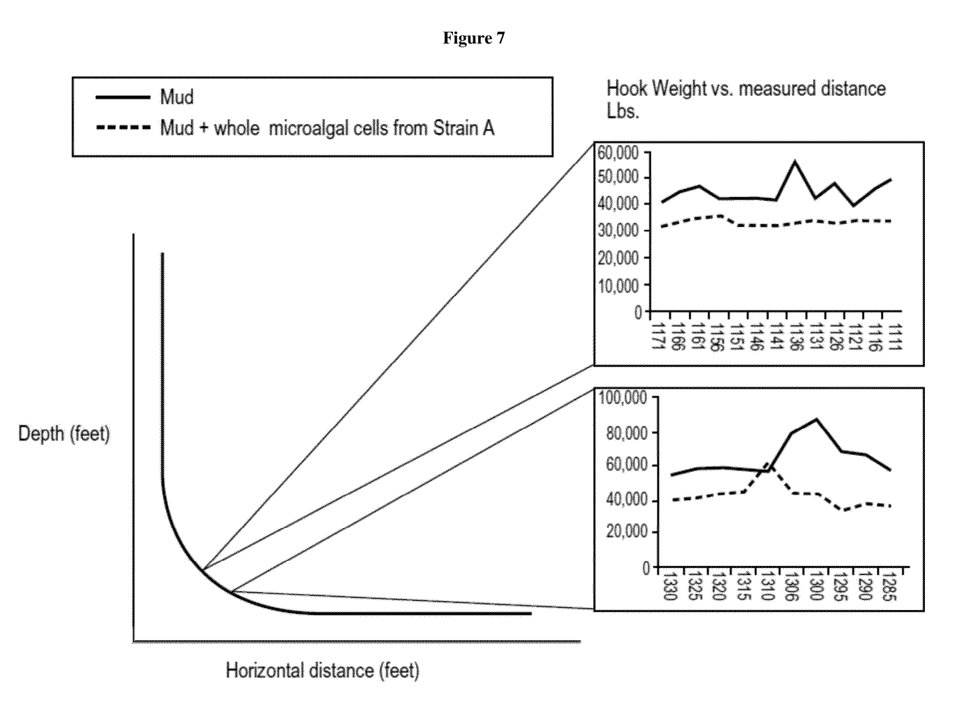

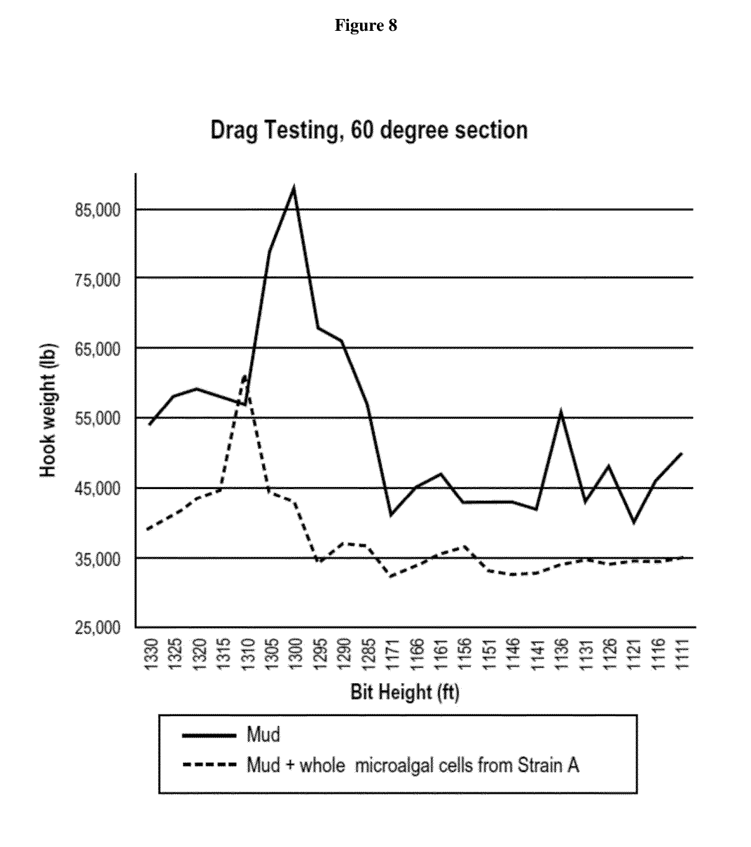

To measure the effect of using whole microalgal cells on the drag encountered by the drill bit, the bottom hole assemblies (BHAs) were pulled out of the aforementioned dug wells and dragged with no rotation along the 45 degree and 60 degree portion of the curve (FIGS. 7 and 8). These drills were either treated with the water based mud alone or with water based mud in combination with the whole microalgal cells. Data was collected on an NOV Totco system connected to a top drive on an oil rig. On average, hook weight was reduced by 27%, with a maximum reduction of 50% in the presence of encapsulated oil.

The changes in drag resulting from the addition of whole microalgal cells to the water based mud are expressed in Table 24. The drag change was computed by taking the difference between the drag when mud alone was used and the drag when mud with whole microalgal cells were used, then dividing that difference by the drag when mud alone was used. These ratios were averaged to arrive at the percent drag reduction at both the 45- and 60-degree portions of the curve.

TABLE 24
Percent Drag Reduction

| | | | Mud + whole | | |
|---|---|---|---|---|---|
| | | Mud | cells | | % Drag |
| | Depth | Drag (lb) | Drag (lb) | Drag change | reduction |
| 60° | 1330 | 54,000 | 39,000 | 0.277777778 | 32% |
| | 1325 | 58,000 | 41,000 | 0.293103448 | |
| | 1320 | 59,000 | 43,500 | 0.262711864 | |
| | 1315 | 58,000 | 44,800 | 0.227586207 | |
| | 1310 | 57,000 | 61,400 | −0.077192982 | |
| | 1305 | 79,000 | 44,400 | 0.437974684 | |
| | 1300 | 88,000 | 43,100 | 0.510227273 | |
| | 1295 | 68,000 | 34,100 | 0.498529412 | |
| | 1290 | 66,000 | 37,000 | 0.439393939 | |
| | 1285 | 57,000 | 36,700 | 0.356140351 | |
| 45° | 1171 | 41,000 | 32,300 | 0.212195122 | 24% |
| | 1166 | 45,000 | 33,800 | 0.248888889 | |
| | 1161 | 47,000 | 35,500 | 0.244680851 | |
| | 1156 | 43,000 | 36,600 | 0.148837209 | |
| | 1151 | 43,000 | 33,000 | 0.23255814 | |
| | 1146 | 43,000 | 32,600 | 0.241860465 | |
| | 1141 | 42,000 | 33,000 | 0.214285714 | |
| | 1136 | 56,000 | 34,000 | 0.392857143 | |
| | 1131 | 43,000 | 34,600 | 0.195348837 | |
| | 1126 | 48,000 | 34,200 | 0.2875 | |
| | 1121 | 40,000 | 34,400 | 0.14 | |
| | 1116 | 46,000 | 34,400 | 0.252173913 | |
| | 1111 | 50,000 | 34,800 | 0.304 | |

As illustrated in FIGS. 7 and 8, the addition of the whole microalgal cells to the water-based mud demonstrated a reduction in hook weight (lb) as a function of bit height. Based on the reduction of hook weight, the use of the mud system with whole microalgal cells led to: (1) a 24% reduction in drag in the 45-degree section of the curve; and (2) a 32% reduction in drag in the 60-degree section of the curve.

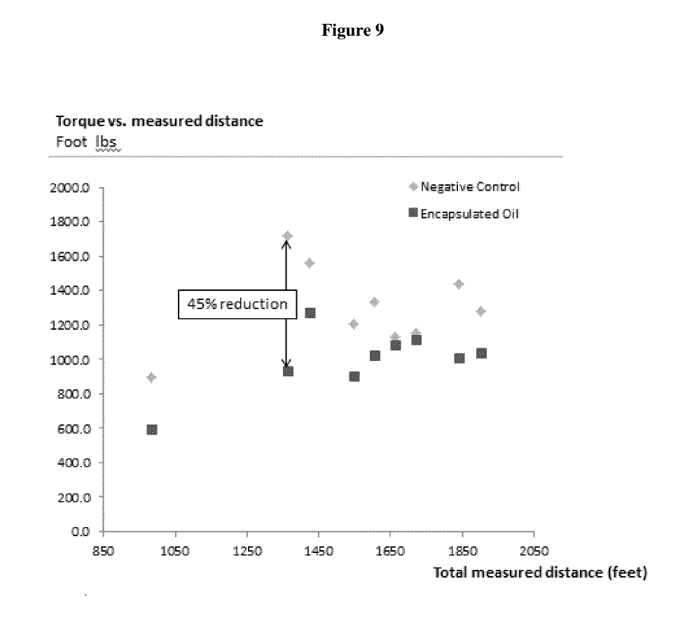

Rotational torque for the top drive was measured by analyzing average torque while rotating off-bottom prior to rotationally drilling in the absence of product. Following encapsulated oil addition, rotational torque was measured at the same points while tripping out. As the pumps were off for the measurements tripping out, a correction factor was applied based on three separate readings done while the pumps were on. On average, rotational torque required to rotate the drill string and bottom hole assembly (BHA) was ˜250 ft*lbs lower when the pumps were on vs. when they were off at the same measured distance (MD), presumably because rotation of the drill bit cones when the pumps were on enabled easier rotation of the entire BHA or because of increased removal of cuttings due to circulation. In the presence of encapsulated oil, rotational torque was reduced by as much as 45% (FIG. 9).

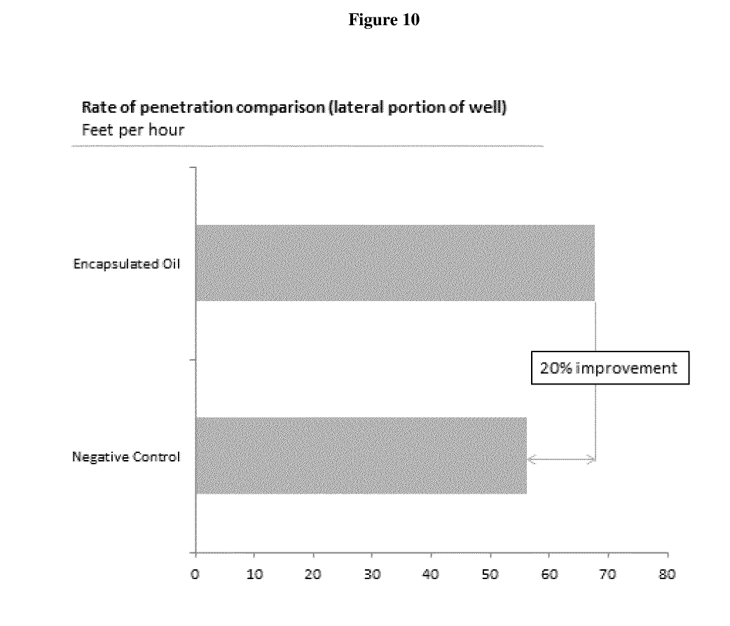

Lateral drilling was performed in the presence and absence of encapsulated oil, rotating at 40-45 RPM and with a weight on bit of 15,000 lbs. Following addition of the encapsulated oil and incubation for 2 hours, rate of penetration (ROP) increased by ˜20% (FIG. 10).

### Example 5

The strains and lubricant in Table 25 below were prepared or obtained and subjected to testing described in Examples 6 and 7.

TABLE 25
Biomass/lubricant

| Biomass Name | Source | Description |
|---|---|---|
| Strain C - oil | Prototheca moriformis | Solvent extracted oil |
| Strain C | Prototheca moriformis | Dried whole cells |
| Strain D | Prototheca moriformis | Dried whole cells |
| Strain E | Auxenochlorella | Dried whole cells |
| | protothecoides | |
| Strain F | Saccharomyces cerevisiae | Dried whole cells |
| Strain G | Rhodoturula glutinis | Dried whole cells |
| Stabil Lube | Ptarmigan Energy | Drilling fluid lubricant |

Strains C was derived from UTEX 1435 classical mutagenized for higher oil production and further transformed with the following plasmid pSZ2533 (SEQ ID NO: 1) for production of triacylglycerides with high oleic acid and low linoleic acid profile. The construct disrupts a single copy of the FATA1 allele while simultaneously expressing a Saccharomyces cerevisiae sucrose invertase and overexpressing a P. moriformis KASII gene (PmKASII). Relevant restriction sites in the construct pSZ2533 FATA13′::CrTUB2:ScSUC2:CvNR::PmUAPA1:PmKASII-CvNR::FATA1 5′ are indicated in lowercase, bold and underlining and are 5′-3′ BspQ 1, Kpn I, Asc I, Mfe I, EcoRV, Spel, AscI, Clal, Sac I, BspQ I, respectively. BspQI sites delimit the 5′ and 3′ ends of the transforming DNA. Bold, lowercase sequences represent FATA1 3′ genomic DNA that permit targeted integration at FATA1 locus via homologous recombination. The C. reinhardtii β-tubulin promoter driving the expression of the yeast sucrose invertase gene is indicated by boxed text. The initiator ATG and terminator TGA for invertase are indicated by uppercase, bold italics while the coding region is indicated in lowercase italics The Chlorella vulgaris nitrate reductase 3′ UTR is indicated by lowercase underlined text followed by the P. moriformis UAPA1 promoter, indicated by boxed italics text. The Initiator ATG and terminator TGA codons of the PmKASII are indicated by uppercase, bold italics, while the remainder of the coding region is indicated by bold italics. The Chlorella protothecoides S106 stearoyl-ACP desaturase transit peptide is located between initiator ATG and the Asc I site. The C. vulgaris nitrate reductase 3′ UTR is again indicated by lowercase underlined text followed by the FATA1 5′ genomic region indicated by bold, lowercase text.

Nucleotide sequence of transforming DNA contained in pSZ2533:

(SEQ ID NO: 1)
gctcttcacccaactcagataataccaatacccctccttctcctcctcatccattcagtacccccccccttctcttcccaaag
cagcaagcgcgtggcttacagaagaacaatcggcttccgccaaagtcgccgagcactgcccgacggcggcgcgcccagcagccc
gcttggccacacaggcaacgaatacattcaatagggggcctcgcagaatggaaggagcggtaaagggtacaggagcactgcgc
acaaggggcctgtgcaggagtgactgactgggcgggcagacggcgcaccgcgggcgcaggcaagcagggaagattgaagcgg
cagggaggaggatgctgattgaggggggcatcgcagtctctcttggacccgggataaggaagcaaatattcggccggttgggttgt
gtgtgtgcacgttttcttcttcagagtcgtgggtgtgcttccagggaggatataagcagcaggatcgaatcccgcgaccagcgtttcc
ccatccagccaaccaccctgtcggtaccctttcttgcgctatgacacttccagcaaaaggtagggcgggctgcgagacggcttcccggc
gctgcatgcaacaccgatgatgcttcgaccccccgaagctccttcggggctgcatgggcgctccgatgccgctccagggcgagcgctgttt
aaatagccaggcccccgattgcaaagacattatagcgagctaccaaagccatattcaaacacctagatcactaccacttctacacaggccac
tcgagcttgtgatcgcactccgctaagggggcgcctcttcctcttcgtttcagtcacaacccgcaaacggcgcgcc [symbol] ctgctgcaggc
cttcctgttcctgctggccggcttcgccgccaagatcagcgcctccatgacgaacgagacgtccgaccgccccctggtgcacttcaccc
ccaacaagggctggatgaacgaccccaacggcctgtggtacgacgagaaggacgccaagtggcacctgtacttccagtacaaccc
gaacgacaccgtctgggggacgcccttgttctggggccacgccacgtccgacgacctgaccaactgggaggaccagcccatcgcca
tcgccccgaagcgcaacgactccggcgccttctccggctccatggtggtggactacaacaacacctccggcttcttcaacgacaccatc
gacccgcgccagcgctgcgtggccatctggacctacaacaccccggagtccgaggagcagtacatctcctacagcctggacggcgg
ctacaccttcaccgagtaccagaagaaccccgtgctggccgccaactccacccagttccgcgacccgaaggtcttctggtacgagccc
tcccagaagtggatcatgaccgcggccaagtcccaggactacaagatcgagatctactcctccgacgacctgaagtcctggaagctg
gagtccgcgttcgccaacgagggcttcctcggctaccagtacgagtgccccggcctgatcgaggtccccaccgagcaggaccccagc
aagtcctactgggtgatgttcatctccatcaaccccggcgccccggccggcggctccttcaaccagtacttcgtcggcagcttcaacggc
acccacttcgaggccttcgacaaccagtcccgcgtggtggacttcggcaaggactactacgccctgcagaccttcttcaacaccgaccc
gacctacgggagcgccctgggcatcgcgtgggcctccaactgggagtactccgccttcgtgcccaccaacccctggcgctcctccatgt
ccctcgtgcgcaagttctccctcaacaccgagtaccaggccaacccggagacggagctgatcaacctgaaggccgagccgatcctg
aacatcagcaacgccggcccctggagccggttcgccaccaacaccacgttgacgaaggccaacagctacaacgtcgacctgtccaa
cagcaccggcaccctggagttcgagctggtgtacgccgtcaacaccacccagacgatctccaagtccgtgttcgcggacctctccctct
ggttcaagggcctggaggaccccgaggagtacctccgcatgggcttcgaggtgtccgcgtcctccttcttcctggaccgcgggaacag
caaggtgaagttcgtgaaggagaacccctacttcaccaaccgcatgagcgtgaacaaccagcccttcaagagcgagaacgacctgt
cctactacaaggtgtacggcttgctggaccagaacatcctggagctgtacttcaacgacggcgacgtcgtgtccaccaacacctacttc
atgaccaccgggaacgccctgggctccgtgaacatgacgacgggggtggacaacctgttctacatcgacaagttccaggtgcgcgag
gtcaag [symbol] caattggcagcagcagctcggatagtatcgacacactctggacgctggtcgtgtgatggactgttgccgccacacttgct
gccttgacctgtgaatatccctgccgcttttatcaaacagcctcagtgtgtttgatcttgtgtgtacgcgcttttgcgagttgctagctgcttgtgct
atttgcgaataccacccccagcatccccttccctcgtttcatatcgcttgcatcccaaccgcaacttatctacgctgtcctgctatccctcagcgc
tgctcctgctcctgctcactgcccctcgcacagccttggtttgggctccgcctgtattctcctggtactgcaacctgtaaaccagcactgcaatg
ctgatgcacgggaagtagtgggatgggaacacaaatggaggatcccgcgtctcgaacagagcgcgcagaggaacgctgaaggtctcgc
ctctgtcgcacctcagcgcggcatacaccacaataaccacctgacgaatgcgcttggttcttcgtccattagcgaagcgtccggttcacaca
cgtgccacgttggcgaggtggcaggtgacaatgatcggtggagctgatggtcgaaacgttcacagcctagggatatcatagcgactgcta
ccccccgaccatgtgccgaggcagaaattatatacaagaagcagatcgcaattaggcacatcgctttgcattatccacacactattcat
cgctgctgcggcaaggctgcagagtgtatttttgtggcccaggagctgagtccgaagtcgacgcgacgagcggcgcaggatccgacc
cctagacgagctctgtcattttccaagcacgcagctaaatgcgctgagaccgggtctaaatcatccgaaaagtgtcaaaatggccgatt
gggttcgcctaggacaatgcgctgcggattcgctcgagtccgctgccggccaaaaggcggtggtacaggaaggcgcacggggccaa
ccctgcgaagccgggggcccgaacgccgaccgccggccttcgatctcgggtgtccccctcgtcaatttcctctctcgggtgcagccacg
aaagtcgtgacgcaggtcacgaaatccggttacgaaaaacgcaggtcttcgcaaaaacgtgagggtttcgcgtctcgccctagctattc
gtatcgccgggtcagacccacgtgcagaaaagcccttgaataacccgggaccgtggttaccgcgccgcctgcaccagggggcttata
taagcccacaccacacctgtctcaccacgcatttctccaactcgcgacttttcggaagaaattgttatccacctagtatagactgccacct
gcaggaccttgtgtcttgcagtttgtattggtcccggccgtcgagctcgacagatctgggctagggttggcctggccgctcggcactcccc
tttagccgcgcgcatccgcgttccagaggtgcgattcggtgtgtggagcattgtcatgcgcttgtgggggtcgttccgtgcgcggcgggtc
cgccatgggcgccgacctgggccctagggtttgttttcgggccaagcgagcccctctcacctcgtcgcccccccgcattccctctctcttg
cagccttgccactagt [symbol] [symbol] [symbol]
ggcgcgcc

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

atcgatagatctcttaaggcagcagcagctcggatagtatcgacacactctggacgctggtcgtgtg
atggactgttgccgccacattgctgccttgacctgtgaatatccctgccgcttttatcaaacagcctcagtgtgtttgatcttgtgtgtacgcgct
tttgcgagttgctagctgcttgtgctatttgcgaataccacccccagcatccccttccctcgtttcatatcgcttgcatcccaaccgcaacttatct
acgctgtcctgctatccctcagcgctgctcctgctcctgctcactgcccctcgcacagccttggtttgggctccgcctgtattctcctggtactg
caacctgtaaaccagcactgcaatgctgatgcacgggaagtagtgggatgggaacacaaatggaaagcttaattaagagctcttgttttcc
agaaggagttgctccttgagcctttcattctcagcctcgataacctccaaagccgctctaattgtggagggggttcgaaccgaatgct
gcgtgaacgggaaggaggaggagaaagagtgagcagggagggattcagaaatgagaaatgagaggtgaaggaacgcatccct
atgcccttgcaatggacagtgtttctggccaccgccaccaagacttcgtgtcctctgatcatcatgcgattgattacgttgaatgcgac
ggccggtcagccccggacctccacgcaccggtgctcctccaggaagatgcgcttgtcctccgccatcttgcagggctcaagctgctc
ccaaaactcttgggcgggttccggacggacggctaccgcgggtgcggccctgaccgccactgttcggaagcagcggcgctgcatg
ggcagcggccgctgcggtgcgccacggaccgcatgatccaccggaaaagcgcacgcgctggagcgcgcagaggaccacagag
aagcggaagagacgccagtactggcaagcaggctggtcggtgccatggcgcgctactaccctcgctatgactcgggtcctcggcc
ggctggcggtgctgacaattcgtttagtggagcagcgactccattcagctaccagtcgaactcagtggcacagtgactccgctcttc

Strain D was derived from UTEX 1435 mutagenized for higher oil production and further transformed with a plasmid to disrupt a stearoyl-ACP desaturase site followed by further mutagenesis. The plasmid was constructed in accordance with methods and sequences described in WO2008/151149, WO2010/063031, WO2010/063032, WO2011/150411, and WO2013/158938 and comprises a C. reinhardtii β-tubulin promoter driving the expression Saccharomyces cerevisiae sucrose invertase gene with a Chlorella protothecoides Ef1 3′ UTR and a Prototheca moriformis endogenous AMT3 promoter driving expression of an exogenous acyl-ACP thioesterase from Cuphea. Wrightii fused to a transit peptide from Prototheca moriformis fatty acid desaturase with a Chlorella vulgaris nitrate reductase 3′ UTR.

Strain E is a Chlorella protothecoides (UTEX 250) strain obtained from the Culture Collection of Alga at the University of Texas (Austin, Tex., USA).

A strain of oleaginous yeast R. glutini (Strain G) and a strain of non-oleaginous yeast S. cerevisiae (Strain F) were cultivated in a nutrient rich complex seed medium (Table 26) at 30° C. and 200 rpm. Primary 250-mL flasks containing 50-60 mL seed medium were inoculated with 1.0-1.5 mL cryopreserved cells (cell bank). At an OD (A₆₀₀) >3, primary flask cultures were used to inoculate secondary flasks containing 60-300 mL seed medium to an initial OD of 0.1-0.2. Strains of yeast were propagated as required by sub-culturing a well-grown inoculum culture (OD >3) into seed medium at OD 0.1-0.2. For production fermentations, the seed culture was cultivated to OD >3 and the seed inoculum volume was typically 10% of the fermentation starting volume (also referred to as the after inoculation volume). The S. cerevisiae strain was propagated through two seed flask stages (primary->secondary->production fermentation-AIV) to prepare the inoculum for the production fermenter. R. glutinis strain was propagated through four seed culture stages (primary->secondary->3^{rd} stage->4^{th} stage->production fermentation) to prepare the inoculum for the production fermenter.

The R. glutinis and S. cerevisiae cultures were cultivated in 15-L lab scale fermenters in a nutrient rich defined medium (Table 26 and Table 27). These fermentations were controlled at a temperature of 30° C., a pH of 5 and dissolved oxygen (DO) >30% of air saturation. The fermentations were aerated at 1.4 volume air/volume medium with automatic control of agitation at 400-1000 rpm as required to control DO. A 13% (w/w) potassium hydroxide solution was used to control pH. The cultures were fed a 71% (w/w dry solids) corn syrup solution on demand in order to maintain residual glucose concentrations in the broth between 0 and 20 g/L. The S. cerevisiae cultures were harvested after cultivation for ˜4 days and 320-460 grams of glucose were consumed per liter after inoculation volume (g/L-AIV). The R. glutinis cultures containing 33% oil were harvested after cultivation for ˜3 days and 230-260 grams of glucose were consumed per liter after inoculation volume (g/L-AIV). The R. glutinis cultures containing 44% oil were harvested after cultivation for 6-7 days and 420-450 grams of glucose were consumed used per liter after inoculation volume (g/L-AIV).

TABLE 26
Composition of seed medium for cultivation of yeast strains.
Medium was prepared by sterilizing in an autoclave at >121° C. for >20
minutes or passing through a sterile 0.2 micron membrane filter.

| | Concentration | |
|---|---|---|
| | (starting fermentation | |
| Medium Components | volume basis) | |
| Peptone | 20 | g/L |
| Yeast Extract | 10 | g/L |
| Thiamine-HCl* | 1.005 | mg/L |
| d-Biotin* | 0.015 | mg/L |
| Cyanocobalimin* | 0.012 | mg/L |
| Calcium Pantothenate* | 0.030 | mg/L |
| p-aminobenzoic acid* | 0.060 | mg/L |
| Glucose* | 20 | g/L |
| Potassium Hydrogen Phthalate* | 5.1 | g/L |

*Sterilized separately and combined aseptically to achieve the final concentration

TABLE 27
Composition of production fermentation
medium for cultivation of yeast strains.
Medium was prepared by sterilizing in an autoclave at >121° C. for >20
minutes or passing through a sterile 0.2 micron membrane filter.

| | Concentration | |
|---|---|---|
| | (starting fermentation | |
| Medium Components | volume basis) | |
| KH₂PO₄ | 10.00 | g/L |
| NaCl | 0.50 | g/L |
| MgSO₄*7H₂O | 3.00 | g/L |
| CaCl₂*2H₂O | 0.50 | g/L |
| (NH₄)₂SO₄ | 10.00 | g/L |
| Antifoam 204 | 0.26 | mL/L |
| (Sigma Chemicals) | | |
| Biotin* | 0.30 | mg/L |
| Calcium Pantothenate* | 3.60 | mg/L |
| Thiamine HCl* | 3.60 | mg/L |
| CuSO4*5H2O* | 1.60 | mg/L |
| COCl2*6H2O* | 4.76 | mg/L |
| ZnSO4*7H2O* | 52.83 | mg/L |
| MnSO4*H2O* | 43.38 | mg/L |
| Na2MoO4*2H2O* | 4.84 | mg/L |
| FeSO4*7H2O* | 55.56 | mg/L |
| 97DE Corn Syrup | 40-60 | g/L |
| (71% dry solids)* | | |

*Sterilized separately and combined aseptically to achieve the final concentration

### Example 6

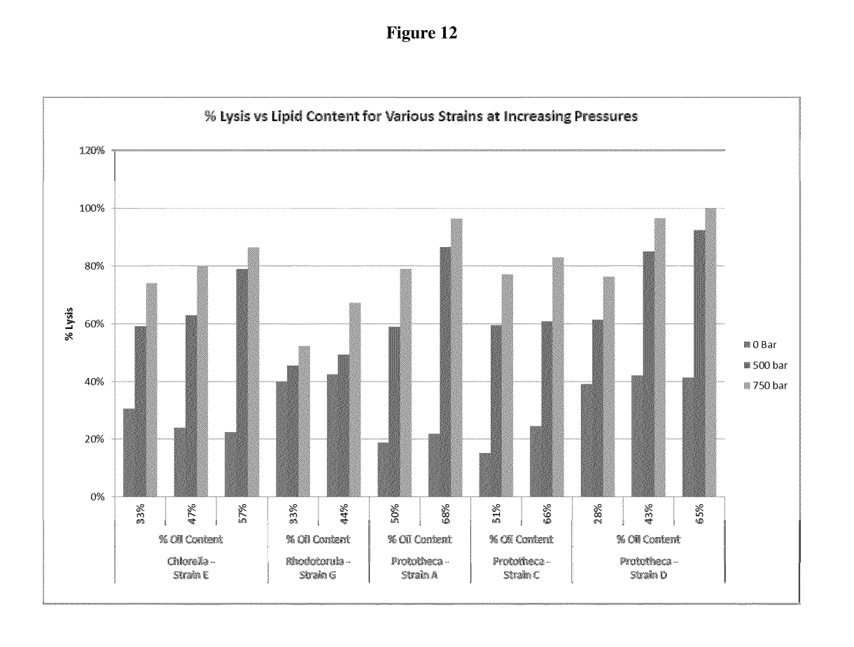

Burst strengths of the biomass in the previous examples were determined by comparing the amount of free oil released for cells of increasing oil content as a function of pressure. Dried biomass was suspended in de-ionized water to 10% total solids, as measured on a Mettler Toledo moisture analyzer by adding 1 g of liquid to a tared glass filter paper and drying at 100° C. The suspension is processed through a Niro Panda lab scale homogenizer unit at the indicated pressures (0, 500, and 750 bar) and collected for free oil analysis. Free oil is extracted from the lysed broth by diluting 0.5 g of sample into 3 mL de-ionized H₂O followed by gentle mixing with a 1:2 hexane and isopropyl alcohol solution for 30 seconds and centrifuged at 12,000 rpm for 5 minutes. The hexane layer containing the oil is transferred with a pipet to a pre-weighed aluminum tray and allowed to evaporate for 60 minutes in a fume hood. The dry oil in the pan is weighed and the % lysis for each sample is determined by dividing the free oil by the total oil available as determined by acid hydrolysis and gas chromatography. Results are summarized in FIG. 12.

### Example 7

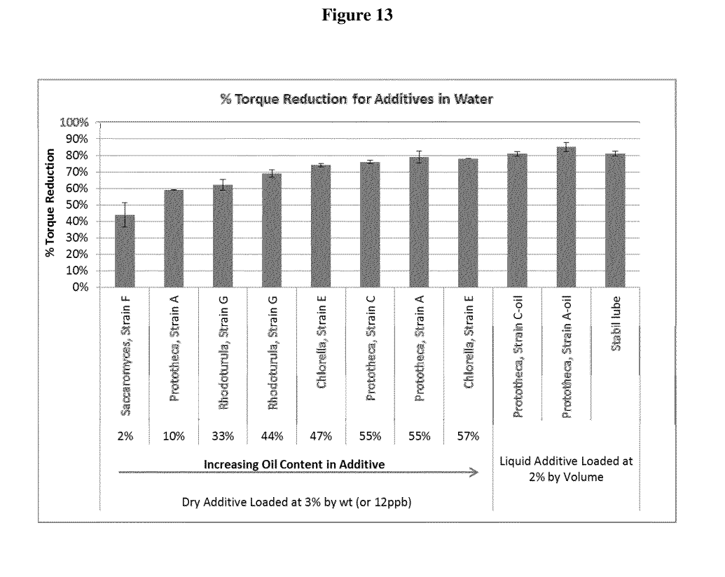

The amounts of additives in water were normalized to strain A containing 55% lipid content. The additives (FIG. 13) were mixed in water to a final concentration of 3% by weight for solid samples (which is 2% total oil by volume for strain A) and 2% by volume for liquid samples. The suspensions were mixed for 3 minutes at low shear using a Hamilton Beach Mixer and then transferred into the sample cup of an OFI Lubricity Meter (model #112-00). For the lubricity coefficient test, 150 in-pounds of force (the equivalent of 5,000 to 10,000 PSI pressure on the intermediate fluid) is applied between two hardened steel surfaces, a block, and a ring rotating at 60 RPM. The % torque reduction is then calculated against the base fluid from the meter reading as described in the equipment manual. Results are shown in FIG. 13.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Informal Sequence Listing

Nucleotide sequence of transforming DNA contained in pSZ2533:

SEQ ID NO: 1

gctcttcacccaactcagataataccaatacccctccttctcctcctcatccattcagtacccccccccttctcttcccaaag
cagcaagcgcgtggcttacagaagaacaatcggcttccgccaaagtcgccgagcactgcccgacggcggcgcgcccagcagccc
gcttggccacacaggcaacgaatacattcaatagggggcctcgcagaatggaaggagcggtaaagggtacaggagcactgcgc
acaaggggcctgtgcaggagtgactgactgggcgggcagacggcgcaccgcgggcgcaggcaagcagggaagattgaagcgg
cagggaggaggatgctgattgaggggggcatcgcagtctctcttggacccgggataaggaagcaaatattcggccggttgggttgt
gtgtgtgcacgttttcttcttcagagtcgtgggtgtgcttccagggaggatataagcagcaggatcgaatcccgcgaccagcgtttcc
ccatccagccaaccaccctgtcggtaccctttcttgcgctatgacacttccagcaaaaggtagggcgggctgcgagacggcttcccggc
gctgcatgcaacaccgatgatgcttcgaccccccgaagctccttcggggctgcatgggcgctccgatgccgctccagggcgagcgctgttt
aaatagccaggcccccgattgcaaagacattatagcgagctaccaaagccatattcaaacacctagatcactaccacttctacacaggccac
tcgagcttgtgatcgcactccgctaagggggcgcctcttcctcttcgtttcagtcacaacccgcaaacggcgcgcc [symbol] ctgctgcaggc
cttcctgttcctgctggccggcttcgccgccaagatcagcgcctccatgacgaacgagacgtccgaccgccccctggtgcacttcaccc
ccaacaagggctggatgaacgaccccaacggcctgtggtacgacgagaaggacgccaagtggcacctgtacttccagtacaaccc
gaacgacaccgtctgggggacgcccttgttctggggccacgccacgtccgacgacctgaccaactgggaggaccagcccatcgcca
tcgccccgaagcgcaacgactccggcgccttctccggctccatggtggtggactacaacaacacctccggcttcttcaacgacaccatc
gacccgcgccagcgctgcgtggccatctggacctacaacaccccggagtccgaggagcagtacatctcctacagcctggacggcgg
ctacaccttcaccgagtaccagaagaaccccgtgctggccgccaactccacccagttccgcgacccgaaggtcttctggtacgagccc
tcccagaagtggatcatgaccgcggccaagtcccaggactacaagatcgagatctactcctccgacgacctgaagtcctggaagctg
gagtccgcgttcgccaacgagggcttcctcggctaccagtacgagtgccccggcctgatcgaggtccccaccgagcaggaccccagc
aagtcctactgggtgatgttcatctccatcaaccccggcgccccggccggcggctccttcaaccagtacttcgtcggcagcttcaacggc
acccacttcgaggccttcgacaaccagtcccgcgtggtggacttcggcaaggactactacgccctgcagaccttcttcaacaccgaccc
gacctacgggagcgccctgggcatcgcgtgggcctccaactgggagtactccgccttcgtgcccaccaacccctggcgctcctccatgt
ccctcgtgcgcaagttctccctcaacaccgagtaccaggccaacccggagacggagctgatcaacctgaaggccgagccgatcctg
aacatcagcaacgccggcccctggagccggttcgccaccaacaccacgttgacgaaggccaacagctacaacgtcgacctgtccaa
cagcaccggcaccctggagttcgagctggtgtacgccgtcaacaccacccagacgatctccaagtccgtgttcgcggacctctccctct
ggttcaagggcctggaggaccccgaggagtacctccgcatgggcttcgaggtgtccgcgtcctccttcttcctggaccgcgggaacag
caaggtgaagttcgtgaaggagaacccctacttcaccaaccgcatgagcgtgaacaaccagcccttcaagagcgagaacgacctgt
cctactacaaggtgtacggcttgctggaccagaacatcctggagctgtacttcaacgacggcgacgtcgtgtccaccaacacctacttc
atgaccaccgggaacgccctgggctccgtgaacatgacgacgggggtggacaacctgttctacatcgacaagttccaggtgcgcgag
gtcaag [symbol] caattggcagcagcagctcggatagtatcgacacactctggacgctggtcgtgtgatggactgttgccgccacacttgct
gccttgacctgtgaatatccctgccgcttttatcaaacagcctcagtgtgtttgatcttgtgtgtacgcgcttttgcgagttgctagctgcttgtgct
atttgcgaataccacccccagcatccccttccctcgtttcatatcgcttgcatcccaaccgcaacttatctacgctgtcctgctatccctcagcgc
tgctcctgctcctgctcactgcccctcgcacagccttggtttgggctccgcctgtattctcctggtactgcaacctgtaaaccagcactgcaatg
ctgatgcacgggaagtagtgggatgggaacacaaatggaggatcccgcgtctcgaacagagcgcgcagaggaacgctgaaggtctcgc
ctctgtcgcacctcagcgcggcatacaccacaataaccacctgacgaatgcgcttggttcttcgtccattagcgaagcgtccggttcacaca
cgtgccacgttggcgaggtggcaggtgacaatgatcggtggagctgatggtcgaaacgttcacagcctagggatatcatagcgactgcta
ccccccgaccatgtgccgaggcagaaattatatacaagaagcagatcgcaattaggcacatcgctttgcattatccacacactattcat
cgctgctgcggcaaggctgcagagtgtatttttgtggcccaggagctgagtccgaagtcgacgcgacgagcggcgcaggatccgacc
cctagacgagctctgtcattttccaagcacgcagctaaatgcgctgagaccgggtctaaatcatccgaaaagtgtcaaaatggccgatt
gggttcgcctaggacaatgcgctgcggattcgctcgagtccgctgccggccaaaaggcggtggtacaggaaggcgcacggggccaa
ccctgcgaagccgggggcccgaacgccgaccgccggccttcgatctcgggtgtccccctcgtcaatttcctctctcgggtgcagccacg
aaagtcgtgacgcaggtcacgaaatccggttacgaaaaacgcaggtcttcgcaaaaacgtgagggtttcgcgtctcgccctagctattc
gtatcgccgggtcagacccacgtgcagaaaagcccttgaataacccgggaccgtggttaccgcgccgcctgcaccagggggcttata
taagcccacaccacacctgtctcaccacgcatttctccaactcgcgacttttcggaagaaattgttatccacctagtatagactgccacct
gcaggaccttgtgtcttgcagtttgtattggtcccggccgtcgagctcgacagatctgggctagggttggcctggccgctcggcactcccc
tttagccgcgcgcatccgcgttccagaggtgcgattcggtgtgtggagcattgtcatgcgcttgtgggggtcgttccgtgcgcggcgggtc
cgccatgggcgccgacctgggccctagggtttgttttcgggccaagcgagcccctctcacctcgtcgcccccccgcattccctctctcttg
cagccttgccactagt

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

atcgatagatctcttaaggcagcagcagctcggatagtatcgacacactctggacgctggtcgtgtg
atggactgttgccgccacacttgctgccttgacctgtgaatatccctgccgcttttatcaaacagcctcagtgtgtttgatcttgtgtgtacgcgct
tttgcgagttgctagctgcttgtgctatttgcgaataccacccccagcatccccttccctcgtttcatatcgcttgcatcccaaccgcaacttatct
acgctgtcctgctatccctcagcgctgctcctgctcctgctcactgcccctcgcacagccttggtttgggctccgcctgtattctcctggtactg
caacctgtaaaccagcactgcaatgctgatgcacgggaagtagtgggatgggaacacaaatggaaagcttaattaagagctcttgttttcc
agaaggagttgctccttgagcctttcattctcagcctcgataacctccaaagccgctctaattgtggagggggttcgaaccgaatgct
gcgtgaacgggaaggaggaggagaaagagtgagcagggagggattcagaaatgagaaatgagaggtgaaggaacgcatccct
atgcccttgcaatggacagtgtttctggccaccgccaccaagacttcgtgtcctctgatcatcatgcgattgattacgttgaatgcgac
ggccggtcagccccggacctccacgcaccggtgctcctccaggaagatgcgcttgtcctccgccatcttgcagggctcaagctgctc
ccaaaactcttgggcgggttccggacggacggctaccgcgggtgcggccctgaccgccactgttcggaagcagcggcgctgcatg
ggcagcggccgctgcggtgcgccacggaccgcatgatccaccggaaaagcgcacgcgctggagcgcgcagaggaccacagag
aagcggaagagacgccagtactggcaagcaggctggtcggtgccatggcgcgctactaccctcgctatgactcgggtcctcggcc
ggctggcggtgctgacaattcgtttagtggagcagcgactccattcagctaccagtcgaactcagtggcacagtgactccgctcttc

1. A drilling fluid for providing delay-released lubrication to a drill bit in a drilling operation, the fluid comprising:
a) a drilling mud and
b) an oleaginous microbial cell;
said fluid capable of providing increasing lubricity during drilling and one or more of
i) at least a 5% reduction in torque to the drill string;
ii) at least a 5% increase in rate of penetration; or
iii) at least a 5% reduction in drag.

2. The drilling fluid of claim 1, wherein the fluid is capable of providing increasing lubricity over at least a 5, 15, 30, 45, or 60 minute time period.

3. The drilling fluid of claim 1 or 2, wherein the fluid is capable of providing at least a 25%, 30%, 35%, 40%, 45%, 50%, 55%, 60%, 65%, 70%, or 75% reduction in torque.

4. The drilling fluid of claim 3, wherein the fluid is capable of providing at least a 60%, 65%, 70%, or 75% reduction in torque.

5. The drilling fluid of any one of claims 1 to 4, wherein the fluid is capable of providing at least a 5%, 10%, 15%, 20%, 25%, 30%, 35%, 40%, or 45% increase in rate of penetration.

6. The drilling fluid of any one of claims 1 to 4, wherein the fluid is capable of providing at least a 5%, 10%, 15%, 20%, 25%, 30%, 35%, 40%, or 45% reduction in drag.

7. The drilling fluid of any one of claims 1 to 6, wherein the microbial cell is in an amount that is 40%, 35%, 30%, 25%, 20%, 15%, 10%, 5%, 3%, 2%, or 1% or less by volume of the drilling fluid.

8. The drilling fluid of any one of claims 1 to 6, wherein the microbial cell is in an amount that is 10% or less by volume of the drilling fluid.

9. The drilling fluid of any one of claims 1 to 6, wherein the microbial cell is in an amount that is 6% or less by volume of the drilling fluid.

10. The drilling fluid of any one of claims 1 to 9, wherein the microbial cell comprises a microalgal cell containing at least 45%, 50%, 55%, 60%, 65%, 70%, 75%, 80%, or 85% oil.

11. The drilling fluid of any one of claims 1 to 10, wherein the microbial cell comprises a whole cell.

12. The drilling fluid of any one of claims 1 to 10, wherein the microbial cell comprises a lysed cell.

13. The drilling fluid of any one of claims 1 to 12, wherein the microbial cell comprises an oleaginous bacteria, yeast, or microalgae.

14. The drilling fluid of any one of claims 1 to 11, wherein the microbial cell is obtained from a heterotrophic oleaginous microalgae.

15. The drilling fluid of any one of claims 1 to 13, wherein the microbial cell is obtained from microalgae cultivated with sugar from corn, sorghum, sugar cane, sugar beet, or molasses as a carbon source.

16. The drilling fluid of any one of claims 1 to 15, wherein the microbial cell is obtained from microalgae cultivated on sucrose.

17. The drilling fluid of any one of claims 1 to 16, wherein the microbial cell is obtained from Parachlorella, Prototheca, or Chlorella.

18. The drilling fluid of any one of claims 1 to 17, wherein the microbial cell is obtained from Prototheca moriformis.

19. The drilling fluid of any one of claims 1 to 18, wherein the microbial cell comprises an oleaginous microalgae having a fatty acid profile of at least 60% C18:1; or at least 50% combined total amount of C10, C12, and C14; or at least 70% combined total amount of C16:0 and C18:1.

20. The drilling fluid of any one of claims 1 to 19, wherein the drilling mud is a water-based mud, a synthetic-based mud, or an oil-based mud.

21. The drilling fluid of any one of claims 1 to 20 further comprising one or more additives selected from the group consisting of an aphron, polymer particle, a thermoset polymer particle, and a nanocomposite particulate.

22. A method for drilling a wellbore in a drilling operation, the method comprising circulating a drilling fluid of any one of claims 1 to 21 through the wellbore.

23. The method of claim 22, wherein the drilling operation is a land-based or an off-shore drilling operation.

24. The method of any one of claims 22 to 23, wherein the drilling operation is selected from the group consisting of completion operations, sand control operations, workover operations, and hydraulic fracturing operations.

25. The method of any one of claims 22 to 24, wherein the wellbore is a vertical or horizontal wellbore.

26. A drilling rig containing a drilling fluid of any one of claims 1 to 21.

27. The drilling rig of claim 26, wherein the fluid is in a drill pipe or mud tank.

28. A method for preparing a drilling fluid for providing delay-released lubrication to a drill bit in a drilling operation, the method comprising mixing a drilling mud with an oleaginous microbial cell to form a drilling fluid capable of increasing lubricity during drilling and one or more of
i) at least a 5% reduction in torque to the drill bit;
ii) at least a 5% increase in rate of penetration; or
iii) at least a 5% reduction in drag.

29. The method of claim 28, wherein the drilling fluid is capable of providing at least a 5%, 10%, 15%, 20%, 25%, 30%, 35%, 40%, or 45% increase in rate of penetration.

30. The method of any one of claims 28 to 29, wherein the drilling fluid is capable of providing at least a 5%, 10%, 15%, 20%, 25%, 30%, 35%, 40%, or 45% reduction in drag.

31. The method of any one of claims 28 to 30, wherein the microbial cell is in an amount that is 40%, 35%, 30%, 25%, 20%, 15%, 10%, 5% or 3% or less by volume of the drilling fluid.

32. The method of any one of claims 28 to 31, wherein the microbial cell is in an amount that is 10% or less by volume of the drilling fluid.

33. The method of any one of claims 28 to 31, wherein the microbial cell is in an amount that is 6% or less by volume of the drilling fluid.

34. The method of any one of claims 28 to 33, wherein the microbial cell is a microalgal cell containing at least 45%, 50%, 55%, 60%, 65%, 70%, 75%, 80%, or 85% oil.

35. The method of any one of claims 28 to 34, wherein the microbial cell comprises a whole cell.

36. The method of any one of claims 28 to 34, wherein the microbial cell comprises a lysed cell.

37. The method of any one of claims 28 to 36, wherein the microbial cell comprises an oleaginous bacteria, yeast, or microalgae.

38. The method of any one of claims 28 to 37, wherein the microbial cell is obtained from a heterotrophic oleaginous microalgae.

39. The method of any one of claims 28 to 38, wherein the microbial cell is obtained from microalgae cultivated with sugar from corn, sorghum, sugar cane, sugar beet, or molasses as a carbon source.

40. The method of any one of claims 28 to 39, wherein the microbial cell is obtained from microalgae cultivated on sucrose.

41. The method of any one of claims 28 to 40, wherein the microbial cell is obtained from Parachlorella, Prototheca, or Chlorella.

42. The method of any one of claims 28 to 41, wherein the microbial cell is obtained from Prototheca moriformis.

43. The method of any one of claims 28 to 42, wherein the microbial cell comprises an oleaginous microalgae having a fatty acid profile of at least 60% C18:1; or at least 50% combined total amount of C10, C12, and C14; or at least 70% combined total amount of C16:0 and C18:1.

44. The method of any one of claims 28 to 43, wherein the drilling mud is a water-based mud, a synthetic-based mud, or an oil-based mud.

45. The method of claim 44 wherein the mud comprises a water-based mud.

46. A lubricant comprising an oleaginous microbial cell, the cell containing at least 45% oil by dry cell weight.

47. The lubricant of claim 46 wherein the cell contains at least 50%, 55%, 60%, 65%, 70%, 75%, or 80% oil by dry cell weight.

48. The lubricant of any one of claims 46 to 47, wherein the lubricant is capable of providing at least a 25%, 30%, 35%, 40%, 45%, 50%, 55%, 60%, 65%, 70%, or 75% reduction in torque.

49. The lubricant of any one of claims 46 to 48, wherein the lubricant is capable of providing at least a 60%, 65%, 70%, or 75% reduction in torque.

50. The lubricant of any one of claims 46 to 49, wherein the fluid is capable of providing at least a 5%, 10%, 15%, 20%, 25%, 30%, 35%, 40%, or 45% increase in rate of penetration.

51. The lubricant of any one of claims 46 to 50, wherein the fluid is capable of providing at least a 5%, 10%, 15%, 20%, 25%, 30%, 35%, 40%, or 45% reduction in drag.

52. The lubricant of any one of claims 46 to 51, wherein the microbial cell comprises a whole cell.

53. The lubricant of any one of claims 46 to 51, wherein the microbial cell comprises a lysed cell.

54. The lubricant of any one of claims 46 to 53, wherein the microbial cell comprises an oleaginous bacteria, yeast, or microalgae.

55. The lubricant of any one of claims 46 to 54, wherein the microbial cell is obtained from a heterotrophic oleaginous microalgae.

56. The lubricant of any one of claims 46 to 55, wherein the microbial cell is obtained from microalgae cultivated with sugar from corn, sorghum, sugar cane, sugar beet, or molasses as a carbon source.

57. The lubricant of any one of claims 46 to 55, wherein the microbial cell is obtained from microalgae cultivated on sucrose.

58. The lubricant of any one of claims 46 to 57, wherein the microbial cell is obtained from Parachlorella, Prototheca, or Chlorella.

59. The lubricant of any one of claims 46 to 58, wherein the microbial cell is obtained from Prototheca moriformis.

60. The lubricant of any one of claims 46 to 59, wherein the microbial cell comprises an oleaginous microalgae having a fatty acid profile of at least 60% C18:1; or at least 50% combined total amount of C10, C12, and C14; or at least 70% combined total amount of C16:0 and C18:1.

61. The lubricant of any one of claims 46 to 60 wherein the lubricant is an extreme pressure lubricant.

62. A metal working fluid comprising a lubricant of any one of claims 46 to 61.

63. The metal working fluid of claim 62, wherein the lubricant is in an amount that is 90%, 80%, 70%, 60%, 50%, 40%, 35%, 30%, 25%, 20%, 15%, 10%, 5%, 3%, 2%, or 1% or less by volume of the fluid.

64. The metal working fluid of any one of claims 62 to 63, wherein the fluid comprises an insoluble oil, soluble oil, semisynthetic, or synthetic metal working fluid.

65. The metal working fluid of any one of claims 62 to 64 further comprising one or more of a surfactant, emulsifier, defoamer, alkaline reserve, anti-mist agent, corrosion inhibitor, biocide, extreme pressure additive, coupling agent, thickener, chelating agent, lubricity agent, humectant, odorant, or dye.

66. The metal working fluid of claim 65 wherein the surfactant comprises an ether, an alkoxylated nonylphenol, or mixtures thereof.

67. The metal working fluid of any one of claims 65 to 66, wherein the emulsifier comprises a hexohydrobenzoic acid, naphthenate, sulfonate, soap, amide, nonionic ethoxylate, an amphoteric, or mixtures thereof.

68. The metal working fluid of any one of claims 65 to 67, wherein the defoamer comprises a silicone, waxy, calcium nitrite, acetate, or mixtures thereof.

69. The metal working fluid of any one of claims 65 to 68, wherein the alkaline reserve comprises an alkanolamine, an alkali hydroxide, or mixtures thereof.

70. The metal working fluid of any one of claims 65 to 69, wherein the anti-mist agent comprises a polybutene, polyacrylate, polyethylene oxide, or mixtures thereof.

71. The metal working fluid of any one of claims 65 to 70, wherein the corrosion inhibitor comprises an amine carboxylate, amine dicarboxylate, boramide, arylsulfonamido acid, sodium borate, sodium molybdate, sodium metasilicate, succinic acid derivative, tolyltriazole, benzotriazole, benzothiazole, thiadiazole, diethanolamine, triethanolamine, nitrite, chlorophenols, cresol, formaldehyde formalin, iodine, phosphate, organic mercurials, phenols, quarternary ammonium compounds, oxammonium, S-triazine compounds, tris-hydroxymethylnitromethane, or mixtures thereof.

72. The metal working fluid of any one of claims 65 to 71, wherein the biocide comprises a triazine, nitromorpholine, polymeric quat, bromonirile, phenol, halogenated carbamate, isothiazolone, or mixtures thereof.

73. The metal working fluid of any one of claims 65 to 72, wherein the extreme pressure additive comprises a sulfurized hydrocarbon, sulfurized fatty acid ester, chlorinated paraffin, chlorinated acid, chlorinated ester, phosphate ester, or mixtures thereof.

74. The metal working fluid of any one of claims 65 to 73, wherein the coupling agent comprises an alcohol, ether, glycol ether, hexylene glycol, or mixtures thereof.

75. The metal working fluid of any one of claims 654 to 74, wherein the thickener comprises a polyether, a polyvinyl alcohol, or mixtures thereof.

76. The metal working fluid of any one of claims 65 to 75, wherein the chelating agent comprises sodium EDTA, a phosphonate, gluconate, or mixtures thereof.

77. The metal working fluid of any one of claims 65 to 76, wherein the lubricity agent comprises an aromatic oil, esters, naphthenic oil, paraffinic oil, polyether glycol, ester, fatty acid ester, glycol ester, block copolymer, or mixtures thereof.

78. The metal working fluid of any one of claims 65 to 77, wherein the humectant comprises a polymeric ether, an ester, or mixtures thereof.

79. The metal working fluid of any one of claims 65 to 78, wherein the odorant comprises an aldehyde.

80. The metal working fluid of any one of claims 65 to 79, wherein the dye comprises an azo dye, a fluorescein, or mixtures thereof.

81. A microtunneling boring machine (MTBM) comprising a lubricant of any one of claims 46 to 60.


SEQUENCE LISTING (1 sequence)

SEQ ID NO: 1 - DNA, 6696 bp, Artificial Sequence
  Description of Artificial Sequence Synthetic polynucleotide
gctcttcacccaactcagataataccaatacccctccttctcctcctcatccattcagtacccccccccttctcttcccaaagcagcaagcgcgtggcttacagaagaacaatcggcttccgccaaagtcgccgagcactgcccgacggcggcgcgcccagcagcccgcttggccacacaggcaacgaatacattcaatagggggcctcgcagaatggaaggagcggtaaagggtacaggagcactgcgcacaaggggcctgtgcaggagtgactgactgggcgggcagacggcgcaccgcgggcgcaggcaagcagggaagattgaagcggcagggaggaggatgctgattgaggggggcatcgcagtctctcttggacccgggataaggaagcaaatattcggccggttgggttgtgtgtgtgcacgttttcttcttcagagtcgtgggtgtgcttccagggaggatataagcagcaggatcgaatcccgcgaccagcgtttccccatccagccaaccaccctgtcggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacggcgcgccatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcatagcgactgctaccccccgaccatgtgccgaggcagaaattatatacaagaagcagatcgcaattaggcacatcgctttgcattatccacacactattcatcgctgctgcggcaaggctgcagagtgtatttttgtggcccaggagctgagtccgaagtcgacgcgacgagcggcgcaggatccgacccctagacgagctctgtcattttccaagcacgcagctaaatgcgctgagaccgggtctaaatcatccgaaaagtgtcaaaatggccgattgggttcgcctaggacaatgcgctgcggattcgctcgagtccgctgccggccaaaaggcggtggtacaggaaggcgcacggggccaaccctgcgaagccgggggcccgaacgccgaccgccggccttcgatctcgggtgtccccctcgtcaatttcctctctcgggtgcagccacgaaagtcgtgacgcaggtcacgaaatccggttacgaaaaacgcaggtcttcgcaaaaacgtgagggtttcgcgtctcgccctagctattcgtatcgccgggtcagacccacgtgcagaaaagcccttgaataacccgggaccgtggttaccgcgccgcctgcaccagggggcttatataagcccacaccacacctgtctcaccacgcatttctccaactcgcgacttttcggaagaaattgttatccacctagtatagactgccacctgcaggaccttgtgtcttgcagtttgtattggtcccggccgtcgagctcgacagatctgggctagggttggcctggccgctcggcactcccctttagccgcgcgcatccgcgttccagaggtgcgattcggtgtgtggagcattgtcatgcgcttgtgggggtcgttccgtgcgcggcgggtccgccatgggcgccgacctgggccctagggtttgttttcgggccaagcgagcccctctcacctcgtcgcccccccgcattccctctctcttgcagccttgccactagtatggccaccgcatccactttctcggcgttcaatgcccgctgcggcgacctgcgtcgctcggcgggctccgggccccggcgcccagcgaggcccctccccgtgcgcgggcgcgccgccgccgccgccgacgccaaccccgcccgccccgagcgccgcgtggtgatcaccggccagggcgtggtgacctccctgggccagaccatcgagcagttctactcctccctgctggagggcgtgtccggcatctcccagatccagaagttcgacaccaccggctacaccaccaccatcgccggcgagatcaagtccctgcagctggacccctacgtgcccaagcgctgggccaagcgcgtggacgacgtgatcaagtacgtgtacatcgccggcaagcaggccctggagtccgccggcctgcccatcgaggccgccggcctggccggcgccggcctggaccccgccctgtgcggcgtgctgatcggcaccgccatggccggcatgacctccttcgccgccggcgtggaggccctgacccgcggcggcgtgcgcaagatgaaccccttctgcatccccttctccatctccaacatgggcggcgccatgctggccatggacatcggcttcatgggccccaactactccatctccaccgcctgcgccaccggcaactactgcatcctgggcgccgccgaccacatccgccgcggcgacgccaacgtgatgctggccggcggcgccgacgccgccatcatcccctccggcatcggcggcttcatcgcctgcaaggccctgtccaagcgcaacgacgagcccgagcgcgcctcccgcccctgggacgccgaccgcgacggcttcgtgatgggcgagggcgccggcgtgctggtgctggaggagctggagcacgccaagcgccgcggcgccaccatcctggccgagctggtgggcggcgccgccacctccgacgcccaccacatgaccgagcccgacccccagggccgcggcgtgcgcctgtgcctggagcgcgccctggagcgcgcccgcctggcccccgagcgcgtgggctacgtgaacgcccacggcacctccacccccgccggcgacgtggccgagtaccgcgccatccgcgccgtgatcccccaggactccctgcgcatcaactccaccaagtccatgatcggccacctgctgggcggcgccggcgccgtggaggccgtggccgccatccaggccctgcgcaccggctggctgcaccccaacctgaacctggagaaccccgcccccggcgtggaccccgtggtgctggtgggcccccgcaaggagcgcgccgaggacctggacgtggtgctgtccaactccttcggcttcggcggccacaactcctgcgtgatcttccgcaagtacgacgagatggactacaaggaccacgacggcgactacaaggaccacgacatcgactacaaggacgacgacgacaagtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctcttgttttccagaaggagttgctccttgagcctttcattctcagcctcgataacctccaaagccgctctaattgtggagggggttcgaaccgaatgctgcgtgaacgggaaggaggaggagaaagagtgagcagggagggattcagaaatgagaaatgagaggtgaaggaacgcatccctatgcccttgcaatggacagtgtttctggccaccgccaccaagacttcgtgtcctctgatcatcatgcgattgattacgttgaatgcgacggccggtcagccccggacctccacgcaccggtgctcctccaggaagatgcgcttgtcctccgccatcttgcagggctcaagctgctcccaaaactcttgggcgggttccggacggacggctaccgcgggtgcggccctgaccgccactgttcggaagcagcggcgctgcatgggcagcggccgctgcggtgcgccacggaccgcatgatccaccggaaaagcgcacgcgctggagcgcgcagaggaccacagagaagcggaagagacgccagtactggcaagcaggctggtcggtgccatggcgcgctactaccctcgctatgactcgggtcctcggccggctggcggtgctgacaattcgtttagtggagcagcgactccattcagctaccagtcgaactcagtggcacagtgactccgctcttc